(19) United States
(12) Patent Application Publication
(10) Pub. No.: US 2022/0064137 A1
(43) Pub. Date: Mar. 3, 2022

(54) COMPOUND, RESIN, COMPOSITION, RESIST PATTERN FORMATION METHOD, CIRCUIT PATTERN FORMATION METHOD, AND PURIFICATION METHOD

(71) Applicant: Mitsubishi Gas Chemical Company, Inc., Tokyo (JP)

(72) Inventors: Takashi SATO, Hiratsuka-shi, Kanagawa (JP); Masatoshi ECHIGO, Chiyoda-ku, Tokyo (JP); Takashi MAKINOSHIMA, Hiratsuka-shi, Kanagawa (JP)

(21) Appl. No.: 17/418,537

(22) PCT Filed: Dec. 25, 2019
(86) PCT No.: PCT/JP2019/050775

§ 371 Date: Jun. 25, 2021

(30) Foreign Application Priority Data
Dec. 28, 2018 (JP) 2018-248508

(51) Int. Cl. C07D 311/82; C08G 8/20; G03F 7/11; G03F 7/038; G03F 7/039

(52) U.S. Cl. CPC C07D 311/82; C08G 8/20; G03F 7/039; G03F 7/038; G03F 7/11

(57) ABSTRACT

The present invention provides a compound comprising a condensed skeleton of an aromatic compound represented by formula (1-1) and an aromatic aldehyde represented by formula (2-1).

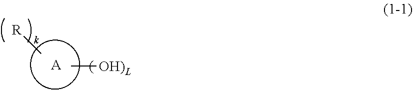

(In the formula (1-1),
A represents an aromatic ring;
R is each independently an alkyl group, an aryl group, an alkenyl group, an alkynyl group, an alkoxy group, a halogen atom, a nitro group, an amino group, a carboxylic acid group, a crosslinkable group, a dissociation group, or a thiol group;
k is an integer of 0 or more; and
L is an integer of 1 or more.)

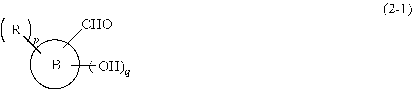

(In the formula (2-1),
B represents an aromatic ring;
R is each independently an alkyl group, an aryl group, an alkenyl group, an alkynyl group, an alkoxy group, a halogen atom, a nitro group, an amino group, a carboxylic acid group, a crosslinkable group, a dissociation group, or a thiol group;
p is an integer of 0 or more; and
q is an integer of 1 or more,
provided that at least one hydroxy group is bonded to a carbon atom adjacent to a carbon atom to which a formyl group is bonded.)

### TECHNICAL FIELD

The present invention relates to a compound, a resin, and a composition containing them, as well as a resist pattern formation method, a circuit pattern formation method, and a purification method. In addition, the present invention particularly relates to a composition to be used for film formation purposes for lithography and film formation purposes for resist, and a film formation method using the same.

### BACKGROUND ART

In recent years, in the production of semiconductor elements and liquid crystal display elements, semiconductors (patterns) and pixels have been rapidly miniaturized due to the advance in lithography technology. As an approach for pixel miniaturization, the exposure light source has been shifted to have a shorter wavelength, in general.

Ultraviolet rays typified by g-ray and i-ray have been used conventionally as the approach for pixel miniaturization, but nowadays, far ultraviolet exposure such as KrF excimer laser (248 nm) and ArF excimer laser (193 nm) is being the center of mass production. Furthermore, the introduction of extreme ultraviolet (EUV) lithography (13.5 nm) is progressing. In addition, electron beam (EB) is also used for forming a fine pattern.

Up to now, typical resist materials are polymer based resist materials capable of forming an amorphous film. As the typical resist materials up to now, polymer based resist materials such as polymethyl methacrylate, polyhydroxy styrene with an acid dissociation group, and polyalkyl methacrylate are known.

Conventionally, a line pattern of about 10 to 100 nm is formed by irradiating a resist thin film fabricated by coating a substrate with a solution of these resist materials with ultraviolet, far ultraviolet, electron beam, extreme ultraviolet or the like.

In addition, lithography using electron beam or extreme ultraviolet has a reaction mechanism different from that of normal photolithography. Furthermore, lithography with electron beam or extreme ultraviolet aims at forming fine patterns of several nm to ten-odd nm. Accordingly, there is a demand for a resist material having higher sensitivity for an exposing source when the resist pattern dimension is reduced. In particular, lithography with extreme ultraviolet is required to further increase sensitivity in terms of throughput.

As a resist material that solves the problems as mentioned above, an inorganic resist material having a metallic element such as titanium, tin, hafnium and zirconium has been proposed (see, for example, Patent Literature 1).

Also, Patent Literature 2 discloses, for example, resist compositions comprising a compound described below. The following compound in Patent Literature 2 is a compound produced by condensing two equivalents of an aromatic compound having a phenolic hydroxy group to one equivalent of an aromatic aldehyde, thereby forming a xanthene ring derived from the aromatic compound having a phenolic hydroxy group.

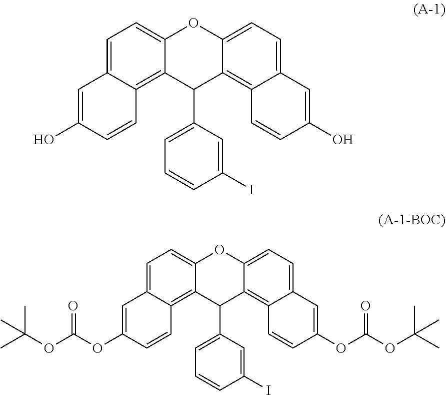 [SMILES: Oc1ccc2c3c(ccc2c1)Oc1ccc2cc(O)ccc2c1C3c1cccc(I)c1 ; CC(C)(C)OC(=O)Oc1ccc2c3c(ccc2c1)Oc1ccc2cc(OC(=O)OC(C)(C)C)ccc2c1C3c1cccc(I)c1]

### CITATION LIST

### Patent Literature

Patent Literature 1: Japanese Patent Laid-Open No. 2015-108781
Patent Literature 2: International Publication No. WO 2016/158168

### SUMMARY OF INVENTION

### Technical Problem

Conventionally developed resist compositions have problems such as many film defects, insufficient sensitivity, insufficient etching resistance, or poor resist pattern.

Also, the aforementioned compound used in the resist compositions disclosed in Patent Literature 2 has high solubility in a safe solvent, good storage stability, and high sensitivity.

However, these resist compositions are required to have further enhanced functions, and in particular, the resist compositions are required to achieve both high resolution and high sensitivity.

In light of the circumstances described above, the present invention has an object to provide a composition that is capable of forming a film achieving both high resolution and high sensitivity, as well as a method for forming a resist pattern and a method for forming an insulating film, using the composition.

### Solution to Problem

The present inventors have, as a result of devoted examinations to solve the problems mentioned above, found out that a specific compound and resin have high solubility in a safe solvent, and are capable of forming a film achieving both high resolution and high sensitivity when the compound and the like are used in a composition for film formation purposes for photography or film formation purposes for resist, leading to completion of the present invention.

More specifically, the present invention is as follows.
[1]

A compound comprising a condensed skeleton of an aromatic compound represented by formula (1-1) and an aromatic aldehyde represented by formula (2-1).

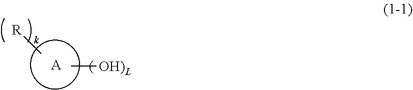

(In the formula (1-1),

A represents an aromatic ring;

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

k is an integer of 0 or more; and

L is an integer of 1 or more.)

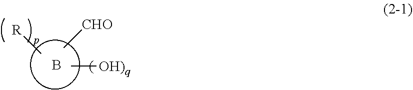

(In the formula (2-1),

B represents an aromatic ring;

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

p is an integer of 0 or more; and

q is an integer of 1 or more,

provided that at least one hydroxy group is bonded to a carbon atom adjacent to a carbon atom to which a formyl group is bonded.)
[2]

The compound according to [1], wherein the condensed skeleton has asymmetry.
[3]

The compound according to [1] or [2], wherein the condensed skeleton is represented by formula (3-1).

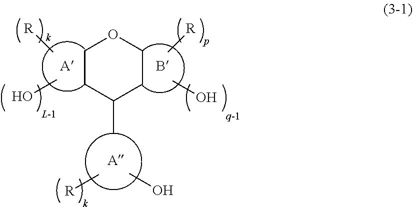

(In the formula (3-1),

A′ and A″ are the same as A in the above formula (1-1);

B′ is the same as B in the above formula (2-1);

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

L is an integer of 1 or more;

p is an integer of 0 or more;

q is an integer of 1 or more; and

k is an integer of 0 or more.)
[4]

The compound according to any of [1] to [3], wherein:

the aromatic compound represented by the formula (1-1) is a compound of the following formula (1-2); and

the aromatic aldehyde represented by the formula (2-1) is a compound of the following formula (2-2).

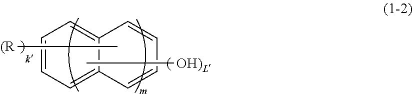

(In the formula (1-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

m is an integer of 0 to 3;

k′ is an integer of 0 to 5 when m=0, an integer of 0 to 7 when m=1, an integer of 0 to 9 when m=2, or an integer of 0 to 11 when m=3; and

L′ is an integer of 1 to 5 when m=0, an integer of 1 to 7 when m=1, an integer of 1 to 9 when m=2, or an integer of 1 to 11 when m=3.)

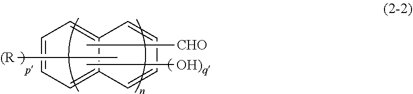

(In the formula (2-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

n is an integer of 0 to 3;

p′ is an integer of 0 to 4 when n=0, an integer of 0 to 6 when n=1, an integer of 0 to 8 when n=2, or an integer of 0 to 10 when n=3; and

q′ is an integer of 1 to 5 when n=0, an integer of 1 to 7 when n=1, an integer of 1 to 9 when n=2, or an integer of 1 to 11 when n=3.)
[5]

The compound according to any of [1] to [4], wherein the condensed skeleton is represented by the following formula (3-2).

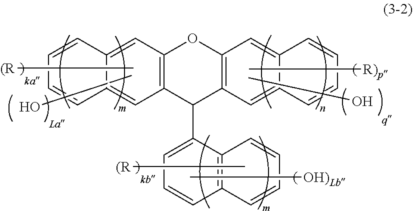

(In the formula (3-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

m is an integer of 0 to 3;

n is an integer of 0 to 3;

ka″ is an integer of 0 to 4 when m=0, an integer of 0 to 6 when m=1, an integer of 0 to 8 when m=2, or an integer of 0 to 10 when m=3;

La″ is an integer of 0 to 4 when m=0, an integer of 0 to 6 when m=1, an integer of 0 to 8 when m=2, or an integer of 0 to 10 when m=3;

kb″ is an integer of 0 to 5 when m=0, an integer of 0 to 7 when m=1, an integer of 0 to 9 when m=2, or an integer of 0 to 11 when m=3;

Lb″ is an integer of 0 to 5 when m=0, an integer of 0 to 7 when m=1, an integer of 0 to 9 when m=2, or an integer of 0 to 11 when m=3;

p″ is an integer of 0 to 4 when n=0, an integer of 0 to 6 when n=1, an integer of 0 to 8 when n=2, or an integer of 0 to 10 when n=3; and

q″ is an integer of 0 to 4 when n=0, an integer of 0 to 6 when n=1, an integer of 0 to 8 when n=2, or an integer of 0 to 10 when n=3.)
[6]

The compound according to any of [1] to [5], wherein the condensed skeleton is represented by the following formula (3-3).

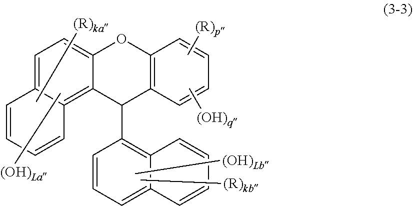

(In the formula (3-3),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

ka″ is an integer of 0 to 6;

La″ is an integer of 0 to 6;

kb″ is an integer of 0 to 7;

Lb″ is an integer of 0 to 7;

p″ is an integer of 0 to 4; and

q″ is an integer of 0 to 4.)
[7]

A compound represented by formula (I).

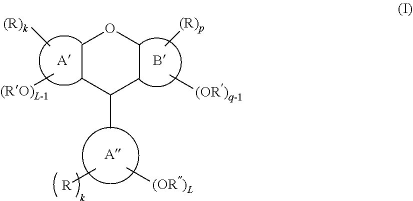

(In the formula (I),

A′ and A″ represent the same aromatic ring;

B′ represents an aromatic ring;

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

L is an integer of 1 or more;

p is an integer of 0 or more;

q is an integer of 1 or more;

k is an integer of 0 or more; and

each —OR′ group is a hydroxy group, a crosslinkable group, or a dissociation group.)
[8]

The compound according to [7], represented by formula (I′).

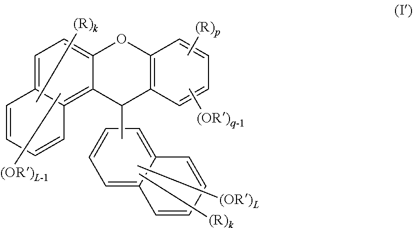

(In the formula (I′),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

L is an integer of 1 or more;

p is an integer of 0 or more;

q is an integer of 1 or more;

k is an integer of 0 or more; and

each —OR′ group is a hydroxy group, a crosslinkable group, or a dissociation group.)
[9]

A method for producing the compound according to any of [1] to [8], the method comprising a step of subjecting a phenol represented by the formula (1-1) and a aromatic aldehyde represented by the formula (2-1) to a condensation reaction, thereby obtaining a skeleton represented by the formula (3-1).
[10]

A resin having a constituent unit derived from the compound according to any of [1] to [8].
[11]

The resin according to [10], wherein the resin has a structure represented by the following formula (4).

[symbol]

L₂-M [symbol] (4)

(In the formula (4), L₂ is a divalent group having 1 to 60 carbon atoms and M is a unit structure derived from the compound according to any of [1] to [5].)
[12]

A composition comprising the compound according to any of [1] to [8] and/or the resin according to [10] or [11].
[13]

The composition according to [12], further comprising a solvent.
[14]

The composition according to [12] or [13], further comprising an acid generating agent.
[15]

The composition according to any of [12] to [14], further comprising a crosslinking agent.
[16]

The composition according to any of [12] to [15], wherein the composition is used in film formation for lithography.
[17]

The composition according to any of [12] to [15], wherein the composition is used in film formation for resist.
[18]

The composition according to any of [12] to [15], wherein the composition is used in resist underlayer film formation.
[19]

The composition according to any of [12] to [15], wherein the composition is used in optical component formation.
[20]

A method for forming a resist pattern, comprising:

a photoresist layer formation step of forming a photoresist layer on a substrate using the composition according to [16] or [17]; and

a development step of irradiating a predetermined region of the photoresist layer formed through the photoresist layer formation step with radiation for development.
[21]

The method for forming a resist pattern according to [20], wherein the resist pattern is an insulating film pattern.
[22]

A method for forming a resist pattern, comprising:

an underlayer film formation step of forming an underlayer film on a substrate using the composition according to [16] or [18];

a photoresist layer formation step of forming at least one photoresist layer on the underlayer film formed through the underlayer film formation step; and

a step of irradiating a predetermined region of the photoresist layer formed through the photoresist layer formation step with radiation for development.
[23]

A method for forming a circuit pattern, comprising:

an underlayer film formation step of forming an underlayer film on a substrate using the composition according to [16] or [18];

an intermediate layer film formation step of forming an intermediate layer film on the underlayer film formed through the underlayer film formation step;

a photoresist layer formation step of forming at least one photoresist layer on the intermediate layer film formed through the intermediate layer film formation step;

a resist pattern formation step of irradiating a predetermined region of the photoresist layer formed through the photoresist layer formation step with radiation for development, thereby forming a resist pattern;

an intermediate layer film pattern formation step of etching the intermediate layer film with the resist pattern formed through the resist pattern formation step as a mask, thereby forming an intermediate layer film pattern;

an underlayer film pattern formation step of etching the underlayer film with the intermediate layer film pattern formed through the intermediate layer film pattern formation step as a mask, thereby forming an underlayer film pattern; and

a substrate pattern formation step of etching the substrate with the underlayer film pattern formed through the underlayer film pattern formation step as a mask, thereby forming a pattern on the substrate.
[24]

A method for purifying the compound according to any of [1] to [8] or the resin according to [10] or [11], comprising:

an extraction step in which extraction is carried out by bringing a solution containing the compound or resin, and an organic solvent that does not inadvertently mix with water into contact with an acidic aqueous solution.

### Advantageous Effects of Invention

According to the present invention, it is possible to provide a compound to be used in a composition that is capable of providing a film achieving both high resolution and high sensitivity in the formation of a resist film, as well as a method for forming a resist pattern and a method for forming an insulating film, using the composition.

### DESCRIPTION OF EMBODIMENTS

Hereinafter, an embodiment of the present invention will be described (hereinafter, may be referred to as the “present embodiment”). The present embodiment is given in order to illustrate the present invention. The present invention is not limited to only the present embodiment.

### [Compound]

A compound of the present embodiment is a compound obtained by subjecting an aromatic compound represented by the formula (1-1) and an aromatic aldehyde represented by the formula (2-1) to a condensation reaction. In addition, the compound of the present embodiment includes a derivative in which a phenolic hydroxy group included in the compound obtained by subjecting an aromatic compound represented by the above formula (1-1) and an aromatic aldehyde represented by the formula (2-1) to a condensation reaction has been derivatized. Here, the phenolic hydroxy group refers to a hydroxy group that is bonded to an aromatic ring.

The aromatic aldehyde represented by the formula (2-1) contains at least one phenolic hydroxy group, and the at least one phenolic hydroxy group is bonded to a carbon atom adjacent to the carbon atom to which the formyl group (aldehyde group) is bonded. Accordingly, the compound obtained by the above condensation reaction comprises a xanthene skeleton formed from the aromatic compound represented by the formula (1-1) and the aromatic aldehyde represented by the formula (2-1).

The compound of the present embodiment may also be referred to as a “compound comprising a condensed skeleton of the aromatic compound represented by the formula (1-1) and the aromatic aldehyde represented by the formula (2-1)”, including the compound obtained by the above condensation reaction and a derivative thereof.

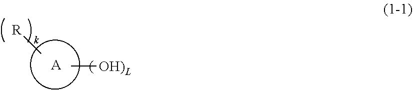

(In the formula (1-1),

A represents an aromatic ring;

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

k is an integer of 0 or more; and

L is an integer of 1 or more.)

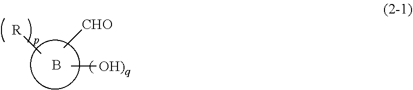

(In the formula (2-1),

B represents an aromatic ring;

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

p is an integer of 0 or more; and

q is an integer of 1 or more,

provided that at least one hydroxy group is bonded to a carbon atom adjacent to a carbon atom to which a formyl group is bonded.)

The condensed skeleton of the aromatic compound represented by the formula (1-1) and the aromatic aldehyde represented by the formula (2-1) includes a xanthene skeleton. It is preferable that this xanthene skeleton is asymmetric with respect to the axis connecting the oxygen atom contained in the pyran ring in xanthene with the methylene carbon contained in the pyran ring. Here, being “asymmetric” indicates that, when the above axis is a mirror surface, the left and right sides separated by the mirror surface do not have a relationship of image and mirror image. In contrast, being “symmetric” indicates that, when the above axis is a mirror surface, the left and right sides separated by the mirror surface have a relationship of image and mirror image.

The compound of the present embodiment is a xanthene compound obtained by a condensation reaction of the aromatic compound represented by the formula (1-1) and the aromatic aldehyde represented by the formula (2-1) or a derivative thereof, and can enhance the film density. This is thought to be because the xanthene skeleton obtained by the condensation reaction of the aromatic compound represented by the formula (1-1) and the aromatic aldehyde represented by the formula (2-1) has asymmetry, which causes the molecules to closely overlap each other and the introduction positions for hydroxy groups to be diverse, resulting in having dense bond formation when the compound becomes a resin.

Due to the enhanced film density, a composition for lithography is obtained that has a high absorption rate of light used in lithography and has high sensitivity. For this reason, a composition suitable for lithography technology is obtained, and it can be used for, without particular limitations, film formation purposes for lithography, for example, resist film formation purposes (that is, a “resist composition”). Furthermore, it can be used for upper layer film formation purposes (that is, a “composition for upper layer film formation”), intermediate layer formation purposes (that is, a “composition for intermediate layer formation”), underlayer film formation purposes (that is, a “composition for underlayer film formation”), and the like. According to the composition of the present embodiment, not only a film having high sensitivity can be formed, but also the composition can impart a good shape to a resist pattern.

A in the formula (1-1) and B in the formula (2-1) each represent an aromatic ring, and examples thereof include, but are not particularly limited to, for example, benzene, naphthalene, anthracene, phenanthrene, tetracene, chrysene, triphenylene, pyrene, pentacene, benzopyrene, coronene, azulene, fluorene, and the like. Among the above, benzene, naphthalene, and anthracene are preferable, and benzene and naphthalene are more preferable.

R in the formula (1-1) and R in the formula (2-1) are each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group.

The alkyl group, the aryl group, the alkenyl group, the alkynyl group, and the alkoxy group each optionally contain an ether bond, a ketone bond, or an ester bond.

Examples of the alkyl group having 1 to 30 carbon atoms in the present embodiment include, but are not particularly limited to, for example, a methyl group, an ethyl group, a n-propyl group, an i-propyl group, a n-butyl group, an i-butyl group, a t-butyl group, a cyclopropyl group, a cyclobutyl group, and the like.

Furthermore, when the alkyl group having 1 to 30 carbon atoms in the present embodiment has a substituent, examples of the alkyl group having 1 to 30 carbon atoms include a methyl group, an ethyl group, a n-propyl group, an i-propyl group, a n-butyl group, an i-butyl group, a t-butyl group, a cyclopropyl group, a cyclobutyl group, and the like that have at least one substituent selected from the group consisting of a halogen atom, a nitro group, an amino group, a thiol group, a hydroxy group, a group in which the hydrogen atom of a hydroxy group is substituted by an acid dissociation group, and the like.

Examples of the aryl group having 6 to 30 carbon atoms in the present embodiment include, but are not particularly limited to, for example, a phenyl group, a naphthalene group, a biphenyl group, and the like.

Furthermore, when the aryl group having 6 to 30 carbon atoms in the present embodiment has a substituent, examples of the aryl group having 6 to 30 carbon atoms include a phenyl group, a naphthalene group, a biphenyl group, and the like that have at least one substituent selected from the group consisting of a halogen atom, a nitro group, an amino group, a thiol group, a hydroxy group, a group in which the hydrogen atom of a hydroxy group is substituted by an acid dissociation group, and the like.

Examples of the alkenyl group having 2 to 30 carbon atoms in the present embodiment include, but are not particularly limited to, for example, a propenyl group, a butenyl group, and the like.

Furthermore, when the alkenyl group having 2 to 30 carbon atoms in the present embodiment has a substituent, examples of the alkenyl group having 2 to 30 carbon atoms include a propenyl group, a butenyl group, and the like that have at least one substituent selected from the group consisting of a halogen atom, a nitro group, an amino group, a thiol group, a hydroxy group, a group in which the hydrogen atom of a hydroxy group is substituted by an acid dissociation group, and the like.

Examples of the alkynyl group having 2 to 30 carbon atoms in the present embodiment include, but are not particularly limited to, for example, a propynyl group, a butynyl group, and the like.

Furthermore, when the alkynyl group having 2 to 30 carbon atoms in the present embodiment has a substituent, examples of the alkynyl group having 2 to 30 carbon atoms include a propynyl group, a butynyl group, and the like that have at least one substituent selected from the group consisting of a halogen atom, a nitro group, an amino group, a thiol group, a hydroxy group, a group in which the hydrogen atom of a hydroxy group is substituted by an acid dissociation group, and the like.

Examples of the alkoxy group having 1 to 30 carbon atoms in the present embodiment include, but are not particularly limited to, for example, a methoxy group, an ethoxy group, a propoxy group, a cyclohexyloxy group, a phenoxy group, a naphthalenoxy group, a biphenyloxy group, and the like.

Furthermore, when the alkoxy group having 1 to 30 carbon atoms in the present embodiment has a substituent, examples of the alkoxy group having 1 to 30 carbon atoms include a methoxy group, an ethoxy group, a propoxy group, a cyclohexyloxy group, a phenoxy group, a naphthalenoxy group, a biphenyloxy group, and the like that have at least one substituent selected from the group consisting of a halogen atom, a nitro group, an amino group, a thiol group, a hydroxy group, a group in which the hydrogen atom of a hydroxy group is substituted by an acid dissociation group, and the like.

The “crosslinkable group” in the present embodiment refers to a group that crosslinks in the presence of a catalyst or without a catalyst. Examples of the crosslinkable group include, but are not particularly limited to, an alkoxy group having 1 to 20 carbon atoms, a group having an allyl group, a group having a (meth)acryloyl group, a group having an epoxy (meth)acryloyl group, a group having a hydroxy group, a group having a urethane (meth)acryloyl group, a group having a glycidyl group, a group having a vinyl containing phenylmethyl group, a group having a group having various alkynyl groups, a group having a carbon-carbon double bond, a group having a carbon-carbon triple bond, a group containing these groups, and the like. Suitable examples of the above group containing these groups include an alkoxy group of the groups described above —OR^{x} (R^{x} is a group having an allyl group, a group having a (meth)acryloyl group, a group having an epoxy (meth)acryloyl group, a group having a hydroxy group, a group having a urethane (meth)acryloyl group, a group having a glycidyl group, a group having a vinyl containing phenylmethyl group, a group having a group having various alkynyl groups, a group having a carbon-carbon double bond, a group having a carbon-carbon triple bond, or a group containing these groups).

Examples of the group having an allyl group include, but are not particularly limited to, for example, a group represented by the following formula (X-1).

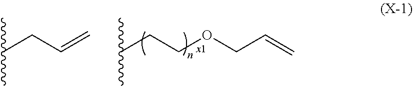

(In the formula (X-1), n^{X1} is an integer of 1 to 5.)

Examples of the group having a (meth)acryloyl group include, but are not particularly limited to, for example, a group represented by the following formula (X-2).

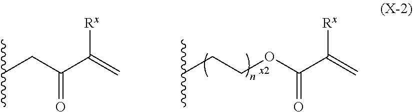

(In the formula (X-2), n^{X2} is an integer of 1 to 5, and R^{X} is a hydrogen atom or a methyl group.)

Examples of the group having an epoxy (meth)acryloyl group include, but are not particularly limited to, for example, a group represented by the following formula (X-3). Here, the epoxy (meth)acryloyl group refers to a group generated through a reaction between an epoxy (meth)acrylate and a hydroxy group.

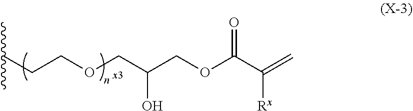

(In the formula (X-3), n^{x3} is an integer of 0 to 5, and R^{X} is a hydrogen atom or a methyl group.)

Examples of the group having a urethane (meth)acryloyl group include, but are not particularly limited to, for example, a group represented by the following formula (X-4).

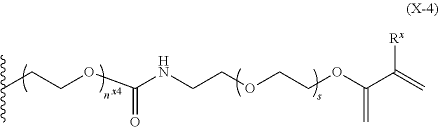

(In the general formula (X-4), n^{x4} is an integer of 0 to 5; s is an integer of 0 to 3; and R^{X} is a hydrogen atom or a methyl group.)

Examples of the group having a hydroxy group include, but are not particularly limited to, for example, a group represented by the following formula (X-5).

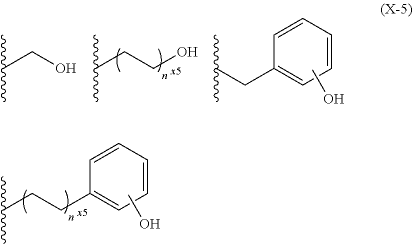

(In the general formula (X-5), n^{x5} is an integer of 1 to 5.)

Examples of the group having a glycidyl group include, but are not particularly limited to, for example, a group represented by the following formula (X-6).

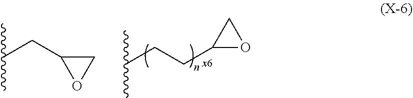

(In the formula (X-6), n^{x6} is an integer of 1 to 5.)

Examples of the group having a vinyl containing phenylmethyl group include, but are not particularly limited to, for example, a group represented by the following formula (X-7).

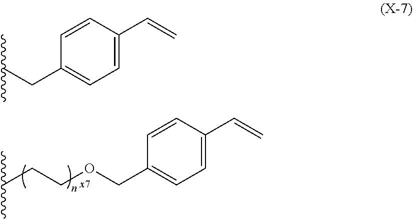

(In the formula (X-7), n^{x7} is an integer of 1 to 5.)

Examples of the group having various alkynyl groups include, but are not particularly limited to, for example, a group represented by the following formula (X-8).

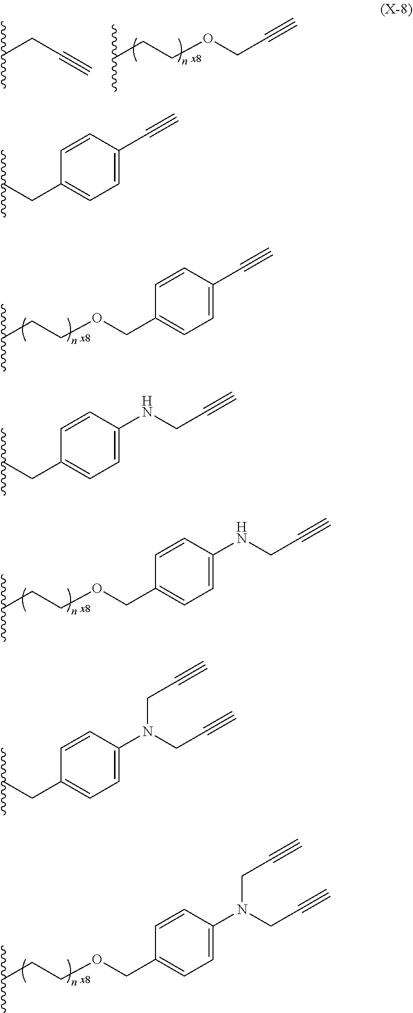

(In the formula (X-8), n^{x8} is an integer of 1 to 5.)

Examples of the above carbon-carbon double bond containing group include, for example, a (meth)acryloyl group, a substituted or unsubstituted vinylphenyl group, a group represented by the following formula (X-9-1), and the like. In addition, examples of the above carbon-carbon triple bond containing group include, for example, a substituted or unsubstituted ethynyl group, a substituted or unsubstituted propargyl group, a group represented by the following formulas (X-9-2) and (X-9-3), and the like.

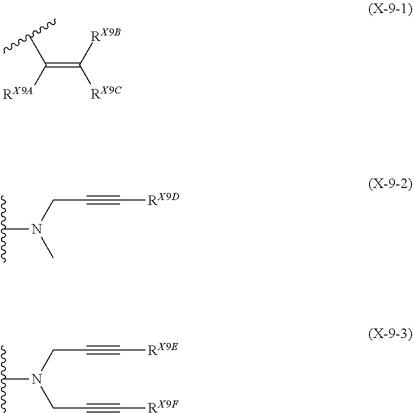

In the above formula (X-9-1), R^{X9A}, R^{X9B} and R^{X9C} are each independently a hydrogen atom or a monovalent hydrocarbon group having 1 to 20 carbon atoms. In the above formulas (X-9-2) and (X-9-3), R^{X9D}, R^{X9E} and R^{X9F} are each independently a hydrogen atom or a monovalent hydrocarbon group having 1 to 20 carbon atoms.

Among the above, a group having a (meth)acryloyl group, an epoxy (meth)acryloyl group, a urethane (meth)acryloyl group, or a glycidyl group, and a group containing a styrene group are preferable; a (meth)acryloyl group, an epoxy (meth)acryloyl group, and a urethane (meth)acryloyl group are more preferable; and a (meth)acryloyl group is still more preferable, from the viewpoint of ultraviolet curability. In addition, from the viewpoint of heat resistance, a group having various alkynyl groups is also preferable.

The “dissociation group” in the present embodiment refers to a group that is dissociated in the presence of a catalyst or without a catalyst. Among the dissociation group, the acid dissociation group refers to a group that is cleaved in the presence of an acid to cause a change into an alkali soluble group or the like.

Examples of the alkali soluble group include, but are not particularly limited to, a phenolic hydroxy group, a carboxyl group, a sulfonic acid group, a hexafluoroisopropanol group, and the like. Among the above, a phenolic hydroxy group and a carboxyl group are preferable, and a phenolic hydroxy group is more preferable, from the viewpoint of the easy availability of an introduction reagent.

The acid dissociation group preferably has the property of causing chained cleavage reaction in the presence of an acid, for achieving pattern formation with high sensitivity and high resolution.

The acid dissociation group is not particularly limited, but can be arbitrarily selected for use from among, for example, those proposed in hydroxystyrene resins, (meth)acrylic acid resins, and the like for use in chemically amplified resist compositions for KrF or ArF.

Specific examples of the acid dissociation group may include those described in International Publication No. WO 2016/158168. Suitable examples of the acid dissociation group include a 1-substituted ethyl group, a 1-substituted n-propyl group, a 1-branched alkyl group, a silyl group, an acyl group, a 1-substituted alkoxymethyl group, a cyclic ether group, an alkoxycarbonyl group, an alkoxycarbonylalkyl group, and the like that have the property of being dissociated by an acid.

Moreover, examples of the substituent in the alkyl group having 1 to 30 carbon atoms and optionally having a substituent, aryl group having 6 to 30 carbon atoms and optionally having a substituent, alkenyl group having 2 to 30 carbon atoms and optionally having a substituent, alkynyl group having 2 to 30 carbon atoms and optionally having a substituent, and alkoxy group having 1 to 30 carbon atoms and optionally having a substituent also include, in addition to the halogen atom, nitro group, amino group, thiol group, hydroxy group, and group in which the hydrogen atom of a hydroxy group is substituted by an acid dissociation group, for example, a cyano group, a heterocyclic group, a linear aliphatic hydrocarbon group, a branched aliphatic hydrocarbon group, a cyclic aliphatic hydrocarbon group, an aryl group, an aralkyl group, an alkoxy group, an amino group, an alkenyl group, an alkynyl group, an acyl group, an alkoxycarbonyl group, an alkyloyloxy group, an aryloyloxy group, an alkylsilyl group, a crosslinkable group, an acid dissociation group, and the like.

It is preferable that the aromatic rings of A in the formula (1-1) and B in the formula (2-1) have at least one hydrogen group on the aromatic rings.

Also, in the present embodiment, the number of substituents (R, OH group, OR′) on the aromatic ring is an integer depending on the type of the aromatic ring. Accordingly, the upper limit value of the index, which refers to the number of substituents on the aromatic ring, is not particularly limited and is determined by the type of the aromatic ring.

In addition, as the condensed skeleton obtained by the condensation reaction between the aromatic compound represented by the formula (1-1) and the aromatic aldehyde represented by the formula (2-1), a compound represented by the formula (3-1) is obtained.

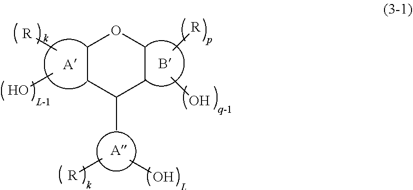

(In the formula (3-1),

A′ and A″ are the same as A in the above formula (1-1);

B′ is the same as B in the above formula (2-1);

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group optionally contain an ether bond, a ketone bond or an ester bond;

L is an integer of 1 or more;

p is an integer of 0 or more;

q is an integer of 1 or more; and

k is an integer of 0 or more.)

In the formula (3-1), R, m, L, p, and q are as defined in R, m, L, p, and q in the above formula (1-1) or the above formula (2-1).

In the present embodiment, it is preferable that L is an integer of 2 or more. When L is an integer of 2 or more, the introduction positions for hydroxy groups in the compound of the present embodiment become diverse, resulting in having dense bond formation when the compound becomes a resin. The upper limit of L is a value that can be arbitrarily determined depending on the number of carbons in A′ and A″, but it is normally 10 or less, and may be 9 or less, 7 or less, or 6 or less.

In the present embodiment, the upper limit of p is a value that can be arbitrarily determined depending on the number of carbons in B′, but it is normally 10 or less, and may be 6 or less, 4 or less, or 2 or less.

In the present embodiment, the upper limit of q is a value that can be arbitrarily determined depending on the number of carbons in B′, but it is normally 10 or less, and may be 6 or less, 4 or less, or 2 or less.

In the present embodiment, the upper limit of k is a value that can be arbitrarily determined depending on the number of carbons in A′ and A″, but it is normally 10 or less, and may be 6 or less, 4 or less, or 2 or less.

It is preferable that the aromatic compound represented by the formula (1-1) is a compound represented by the formula (1-2) from the viewpoint of reactivity.

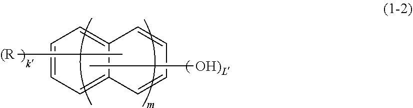

(In the formula (1-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

m is an integer of 0 to 3;

k′ is an integer of 0 to 5 when m=0, an integer of 0 to 7 when m=1, an integer of 0 to 9 when m=2, or an integer of 0 to 11 when m=3; and

L′ is an integer of 1 to 5 when m=0, an integer of 1 to 7 when m=1, an integer of 1 to 9 when m=2, or an integer of 1 to 11 when m=3.)

The sum of k′ and L′ may be:

an integer of 1 to 5 when m=0, an integer of 1 to 7 when m=1, an integer of 1 to 9 when m=2, or an integer of 1 to 11 when m=3;

an integer of 1 to 4 when m=0, an integer of 1 to 6 when m=1, an integer of 1 to 8 when m=2, or an integer of 1 to 10 when m=3; or

an integer of 1 to 3 when m=0, an integer of 1 to 5 when m=1, an integer of 1 to 7 when m=2, or an integer of 1 to 9 when m=3.

It is preferable that the aromatic aldehyde represented by the formula (2-1) is an aromatic aldehyde represented by the formula (2-2) from the viewpoint of reactivity.

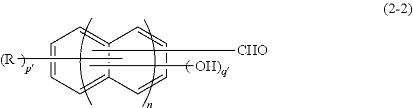

(In the formula (2-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

n is an integer of 0 to 3;

p′ is an integer of 0 to 4 when n=0, an integer of 0 to 6 when n=1, an integer of 0 to 8 when n=2, or an integer of 0 to 10 when n=3; and

q′ is an integer of 1 to 5 when n=0, an integer of 1 to 7 when n=1, an integer of 1 to 9 when n=2, or an integer of 1 to 11 when n=3.)

The sum of p′ and q′ may be:

an integer of 1 to 5 when m=0, an integer of 1 to 7 when m=1, an integer of 1 to 9 when m=2, or an integer of 1 to 11 when m=3;

an integer of 1 to 4 when m=0, an integer of 1 to 6 when m=1, an integer of 1 to 8 when m=2, or an integer of 1 to 10 when m=3; or

an integer of 1 to 3 when m=0, an integer of 1 to 5 when m=1, an integer of 1 to 7 when m=2, or an integer of 1 to 9 when m=3.

At least one of R in the formula (1-1) or formula (2-1) is preferably a halogen atom, a nitro group, a crosslinkable group, or a thiol group, is more preferably a halogen atom, and is still more preferably at least one selected from the group consisting of chlorine, bromine, and iodine, from the viewpoint of enhancing film density.

It is preferable that the condensed skeleton in the present embodiment is a compound represented by the formula (3-2) from the viewpoint of ease of production.

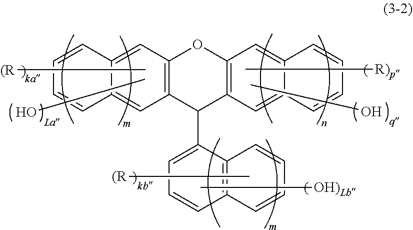

(In the formula (3-2),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;

the alkyl group, the aryl group, the alkenyl group, the alkynyl group, and the alkoxy group each optionally contain an ether bond, a ketone bond, or an ester bond;

m is an integer of 0 to 3;

n is an integer of 0 to 3;

ka″ is an integer of 0 to 4 when m=0, an integer of 0 to 6 when m=1, an integer of 0 to 8 when m=2, or an integer of 0 to 10 when m=3;

La″ is an integer of 0 to 4 when m=0, an integer of 0 to 6 when m=1, an integer of 0 to 8 when m=2, or an integer of 0 to 10 when m=3;

kb″ is an integer of 0 to 5 when m=0, an integer of 0 to 7 when m=1, an integer of 0 to 9 when m=2, or an integer of 0 to 11 when m=3;

Lb″ is an integer of 0 to 5 when m=0, an integer of 0 to 7 when m=1, an integer of 0 to 9 when m=2, or an integer of 0 to 11 when m=3;

p″ is an integer of 0 to 4 when n=0, an integer of 0 to 6 when n=1, an integer of 0 to 8 when n=2, or an integer of 0 to 10 when n=3; and

q″ is an integer of 0 to 4 when n=0, an integer of 0 to 6 when n=1, an integer of 0 to 8 when n=2, or an integer of 0 to 10 when n=3.)

The sum of ka″ and La″ may be:

an integer of 0 to 4 when m=0, an integer of 0 to 6 when m=1, an integer of 0 to 8 when m=2, or an integer of 0 to 10 when m=3;

an integer of 0 to 3 when m=0, an integer of 0 to 5 when m=1, an integer of 0 to 7 when m=2, or an integer of 0 to 9 when m=3; or

an integer of 0 to 2 when m=0, an integer of 0 to 4 when m=1, an integer of 0 to 6 when m=2, or an integer of 0 to 8 when m=3.

The sum of kb″ and Lb″ may be:

an integer of 0 to 5 when m=0, an integer of 0 to 7 when m=1, an integer of 0 to 9 when m=2, or an integer of 0 to 11 when m=3;

an integer of 1 to 5 when m=0, an integer of 1 to 7 when m=1, an integer of 1 to 9 when m=2, or an integer of 1 to 11 when m=3;

an integer of 1 to 4 when m=0, an integer of 1 to 6 when m=1, an integer of 1 to 8 when m=2, or an integer of 1 to 10 when m=3; or

an integer of 1 to 3 when m=0, an integer of 1 to 5 when m=1, an integer of 1 to 7 when m=2, or an integer of 1 to 10 when m=3.

The sum of p″ and q″ may be:

an integer of 0 to 4 when n=0, an integer of 0 to 6 when n=1, an integer of 0 to 8 when n=2, or an integer of 0 to 10 when n=3;

an integer of 0 to 3 when n=0, an integer of 0 to 5 when n=1, an integer of 0 to 7 when n=2, or an integer of 0 to 9 when n=3; or

an integer of 0 to 2 when n=0, an integer of 0 to 4 when n=1, an integer of 0 to 6 when n=2, or an integer of 0 to 8 when n=3.

Here, the compound represented by the formula (3-2) comprises a structure represented by the following formula (A-0) as an aromatic ring moiety. The aromatic ring represented by the formula (A-0) is a structure that schematically represents an aromatic ring and includes isomeric structures. Specific examples of the aromatic ring represented by the formula (A-0) include the structures shown in (A-1).

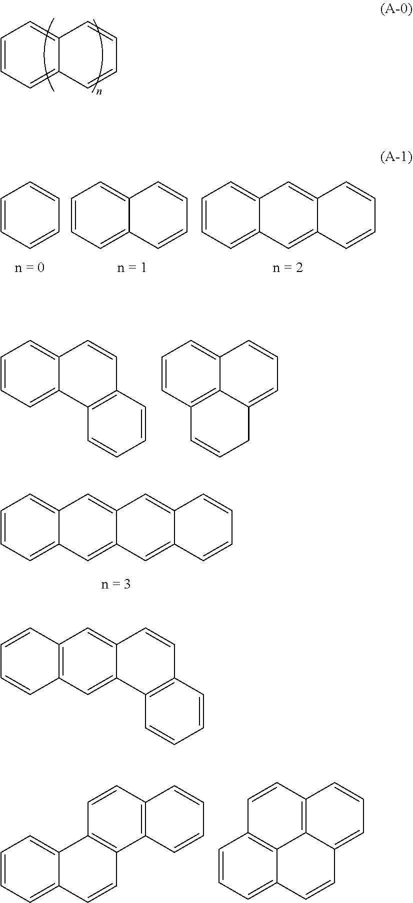

It is preferable that the condensed skeleton in the present embodiment is represented by the following formula (3-3). A compound comprising a condensed skeleton represented by the following formula (3-3) tends to have dense bond formation when it becomes a resin, resulting in a composition for lithography having an enhanced film density, a high absorption rate of light used in lithography, and high sensitivity.

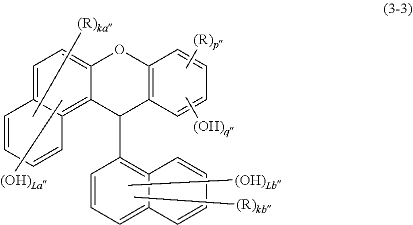

In the formula (3-3),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group,

the alkyl group, the aryl group, the alkenyl group, the alkynyl group, and the alkoxy group each optionally contain an ether bond, a ketone bond, or an ester bond,

ka″ is an integer of 0 to 6,

La″ is an integer of 0 to 6,

kb″ is an integer of 0 to 7,

Lb″ is an integer of 0 to 7,

p″ is an integer of 0 to 4, and

q″ is an integer of 0 to 4.

The compound of the present embodiment is also a compound represented by the formula (I).

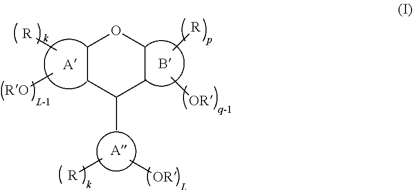

In the formula (I),

A′ and A″ represent the same aromatic ring,

B′ represents an aromatic ring,

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group or a thiol group,

the alkyl group, the aryl group, the alkenyl group, the alkynyl group, and the alkoxy group each optionally contain an ether bond, a ketone bond or an ester bond,

L is an integer of 1 or more,

p is an integer of 0 or more,

q is an integer of 1 or more,

k is an integer of 0 or more, and

each —OR′ group is a hydroxy group, a crosslinkable group, or a dissociation group.

A′ and A″ in the formula (I) are the same aromatic ring, but the bonding form with the adjacent ring and the substituents may not be the same.

As for the aromatic rings of A′, A″, and B′ in the formula (I), the same aromatic rings as the aromatic rings in the formula (1-1) and the formula (1-2) can be exemplified, and the same preferable aromatic rings may be mentioned.

In addition, specific examples of the aromatic rings of A′, A″, and B′ in the formula (I) may include a structure represented by the following formula (A-0). The aromatic ring represented by the formula (A-0) is a structure that schematically represents an aromatic ring, and includes isomeric structures. Examples of the aromatic ring represented by the formula (A-0) include, specifically, the structures shown in (A-1).

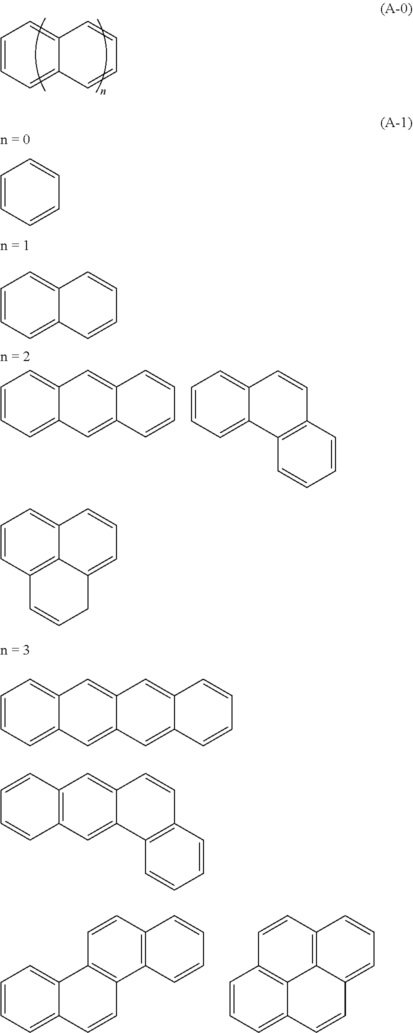

As for the alkyl group having 1 to 30 carbon atoms and optionally having a substituent, aryl group having 6 to 30 carbon atoms and optionally having a substituent, alkenyl group having 2 to 30 carbon atoms and optionally having a substituent, alkynyl group having 2 to 30 carbon atoms and optionally having a substituent, alkoxy group having 1 to 30 carbon atoms and optionally having a substituent, crosslinkable group, and dissociation group of R in the formula (I), the same groups as the alkyl group having 1 to 30 carbon atoms and optionally having a substituent, aryl group having 6 to 30 carbon atoms and optionally having a substituent, alkenyl group having 2 to 30 carbon atoms and optionally having a substituent, alkynyl group having 2 to 30 carbon atoms and optionally having a substituent, alkoxy group having 1 to 30 carbon atoms and optionally having a substituent, crosslinkable group, and dissociation group in the formula (1-1) and the formula (1-2) can be exemplified, and the same preferable groups may be mentioned.

Each —OR′ group in the formula (I) is a hydroxy group (—OH), a crosslinkable group, or a dissociation group. As for the crosslinkable group, the same groups as the crosslinkable groups in the formula (1-1) and the formula (1-2) can be exemplified, and the same preferable groups may be mentioned. As for the dissociation group, the same groups as the dissociation groups in the formula (1-1) and the formula (1-2) can be exemplified, and the same preferable groups may be mentioned.

The compound represented by the formula (I) comprises a xanthene skeleton (ring A′-pyran ring-ring B′). It is preferable that this xanthene skeleton is asymmetric with respect to the axis connecting the oxygen atom contained in the pyran ring in xanthene with the methylene carbon contained in the pyran ring. Here, being “asymmetric” indicates that when the above axis is a mirror surface, the left and right sides separated by the mirror surface do not have a relationship of image and mirror image. In contrast, being “symmetric” indicates that when the above axis is a mirror surface, the left and right sides separated by the mirror surface have a relationship of image and mirror image.

It is preferable that the compound represented by the formula (I) of the present embodiment is the compound represented by the formula (3-2), and at this time, the —OH groups in the compound represented by the formula (3-2) may each be a crosslinkable group and/or a dissociation group.

It is preferable that the compound of the present embodiment comprise a condensed skeleton with each of the following structures. The following structures correspond to “ring A′-pyran ring (-ring A”)-ring B′″ in the formula (I) of the present embodiment.

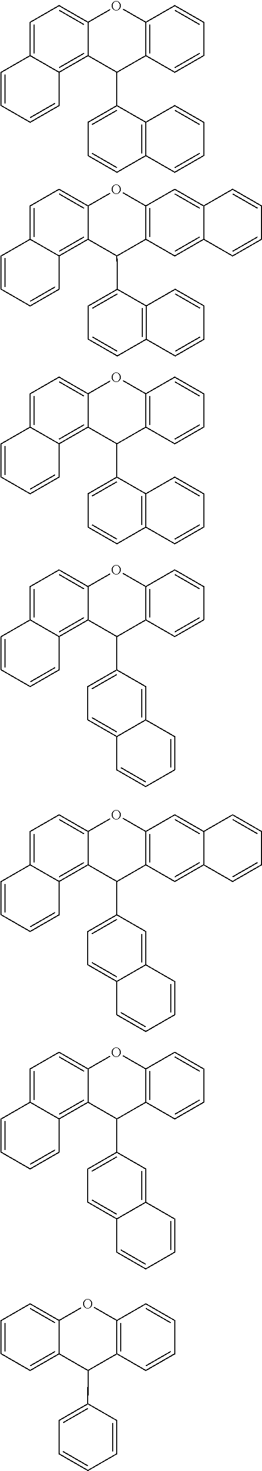

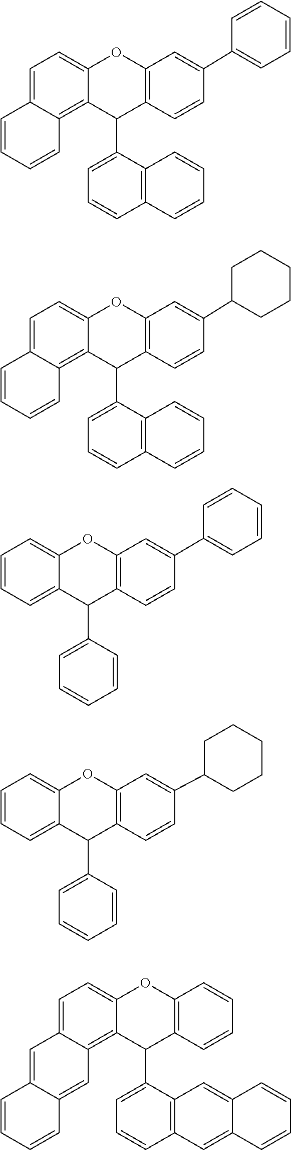

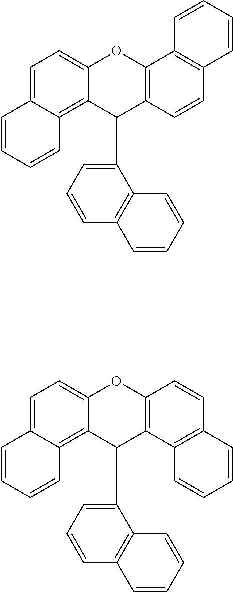 [SMILES: c1ccc2c(C3c4ccc5ccccc5c4Oc4ccc5ccccc5c43)cccc2c1 ; c1ccc2c(C3c4c(ccc5ccccc45)Oc4ccc5ccccc5c43)cccc2c1]

It is more preferable that the compound of the present embodiment comprise a condensed skeleton with each of the following structures.

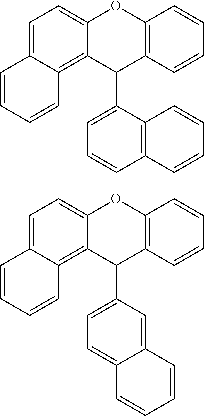

When the compound of the present embodiment comprises each of the condensed skeletons described above, it is preferable that it is represented by the formula (I′).

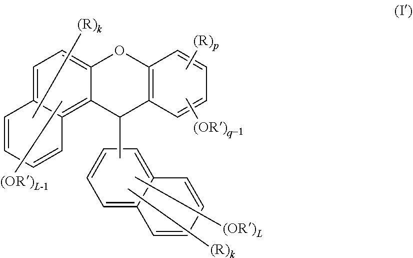

In the formula (I′), R, L, p, q, k, and —OR′ are as defined in R, L, p, q, k, and —OR′ in the formula (I), and can be the same preferable groups and numbers.

Specifically, in the formula (I′),

R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group,

the alkyl group, the aryl group, the alkenyl group, the alkynyl group, and the alkoxy group optionally contain an ether bond, a ketone bond, or an ester bond,

L is an integer of 1 or more,

p is an integer of 0 or more,

q is an integer of 1 or more,

k is an integer of 0 or more, and

each —OR′ group is a hydroxy group, a crosslinkable group or a dissociation group.

The compound represented by the formula (I′) is preferably represented by formula (I″).

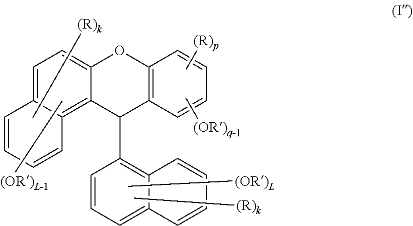

In the formula (I″), R, L, p, q, k, and —OR′ are as defined in R, L, p, q, k, and —OR′ in the formula (I), and can be the same preferable groups and numbers.

In addition, the compound represented by the formula (I″) is preferably represented by the following formulas.

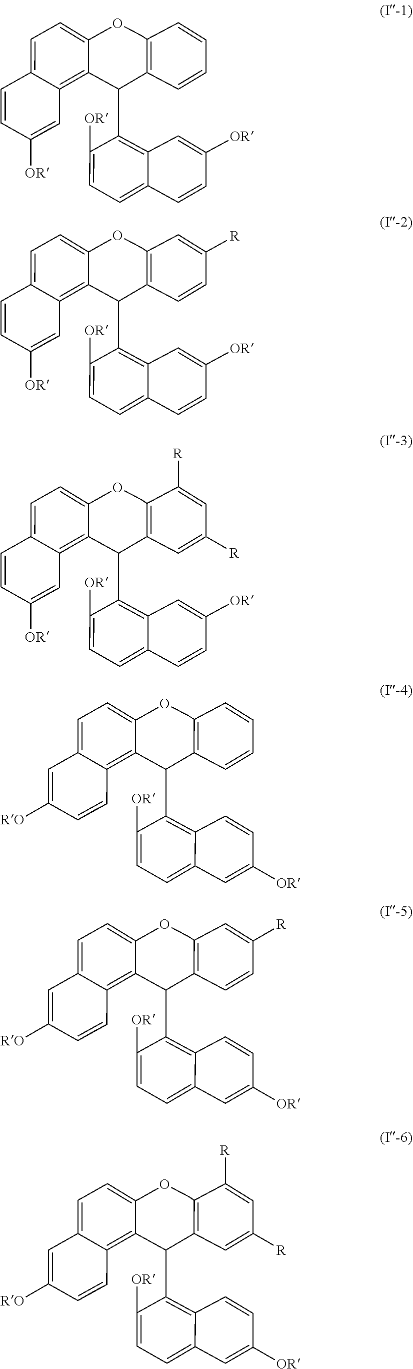

In the formulas (I″-1) to (I″-6), R and —OR′ are as defined in R and —OR′ in the formula (I), and can be the same preferable groups.

Each R in the formulas (I″-1) to (I″-6) is preferably an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, or a halogen atom, and is more preferably an aryl group having 6 to 30 carbon atoms and optionally having a substituent or a halogen atom.

The compound of the present embodiment is specifically exemplified in the following. However, the compound of the present embodiment is not limited to them.

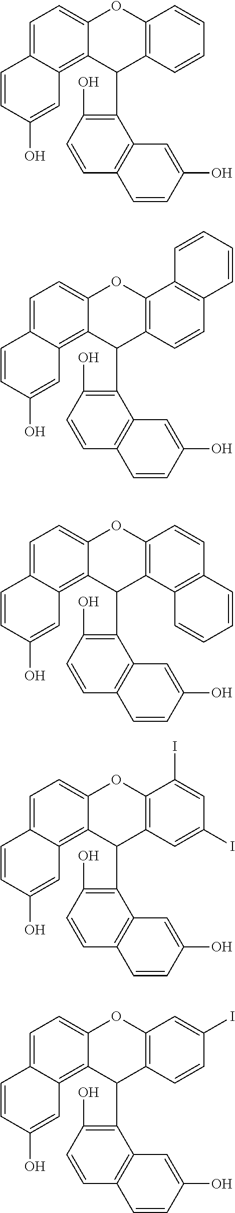

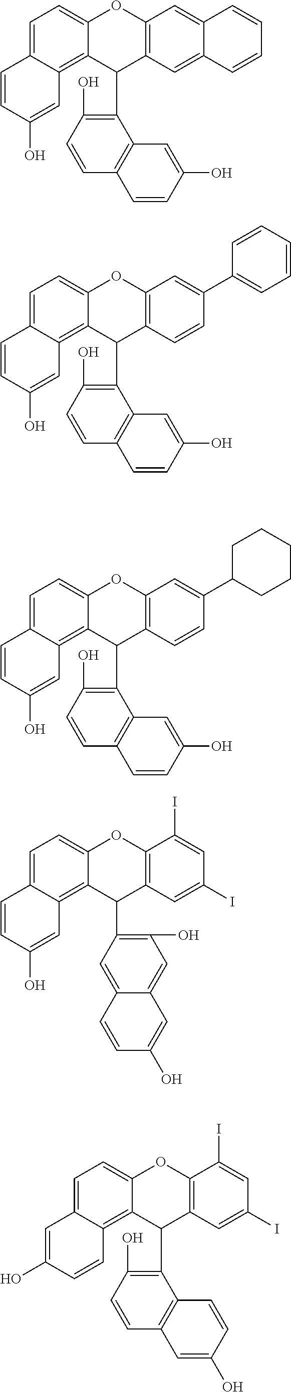

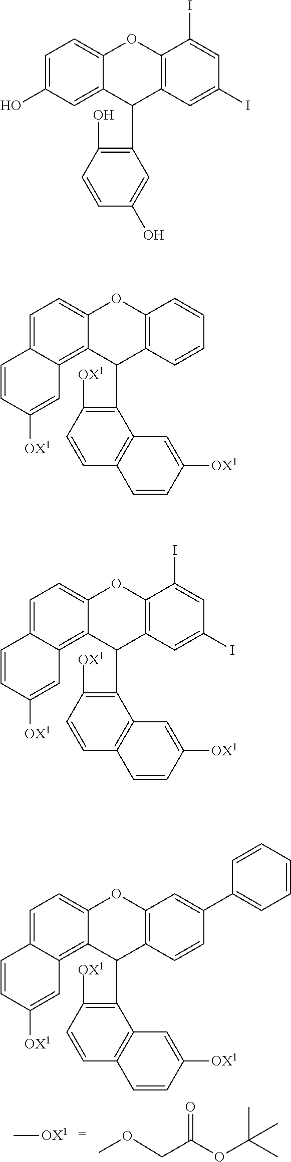

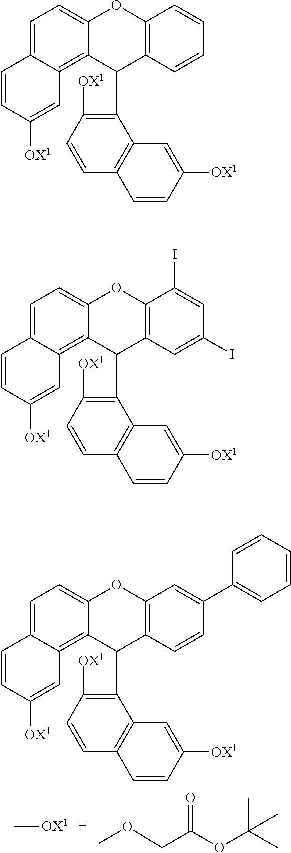

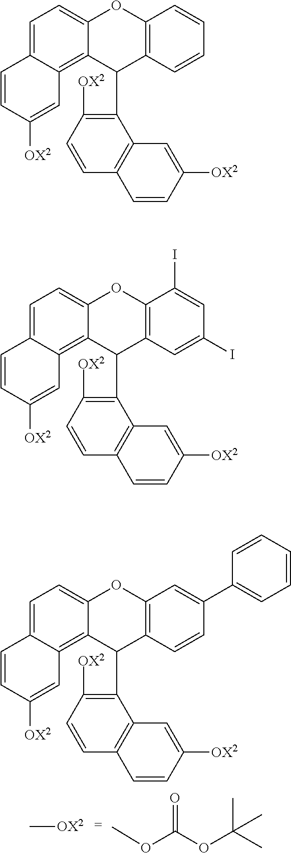

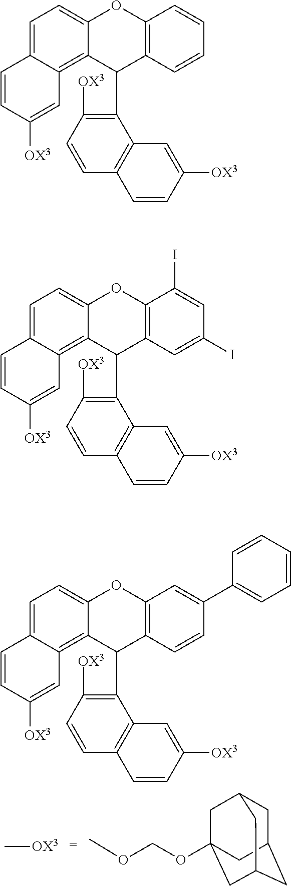

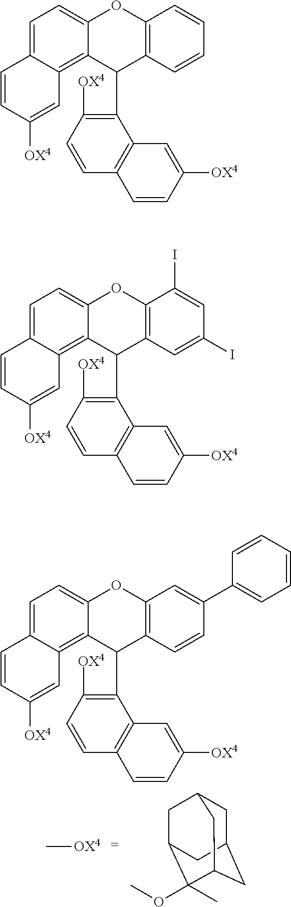

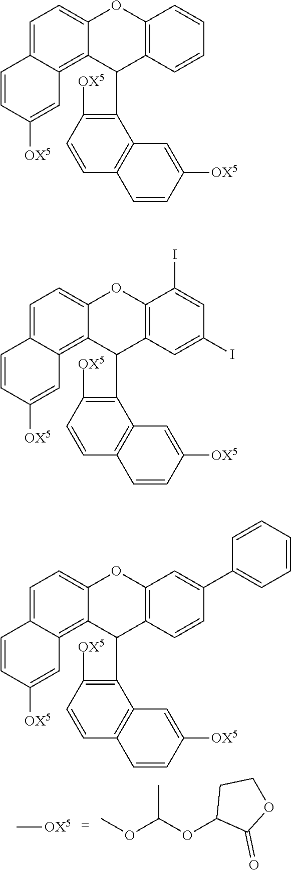

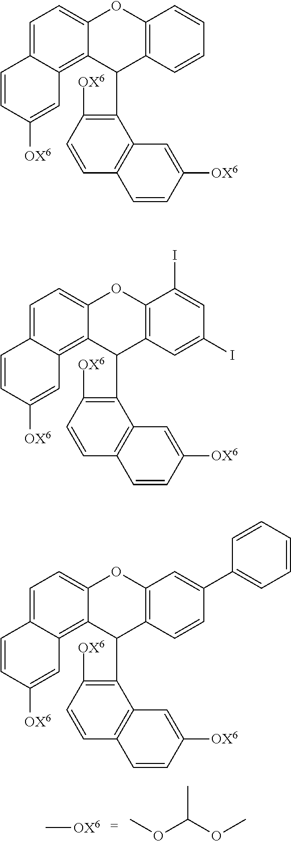

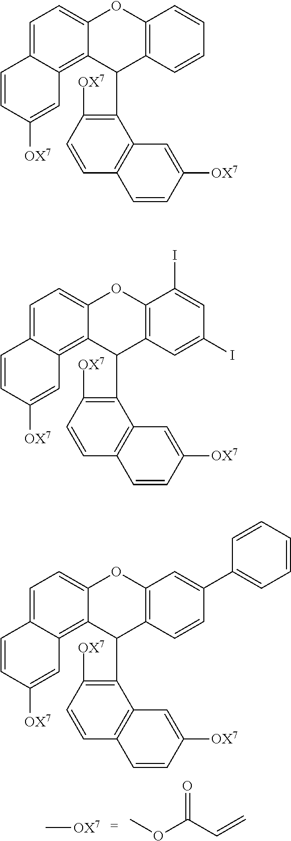

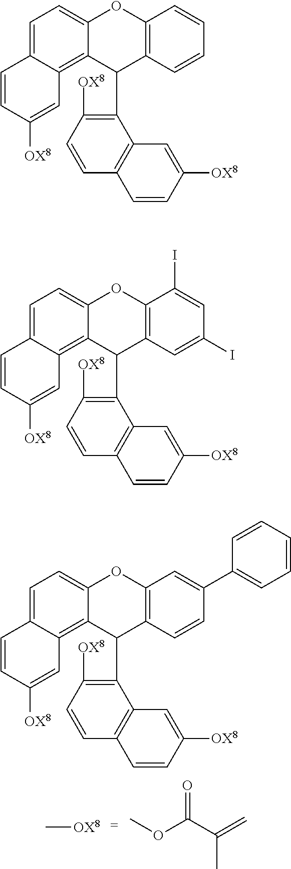

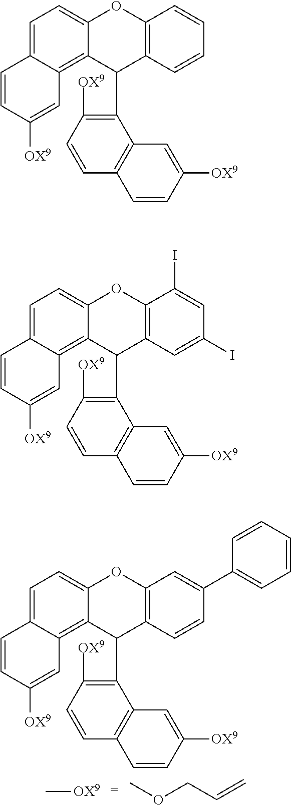

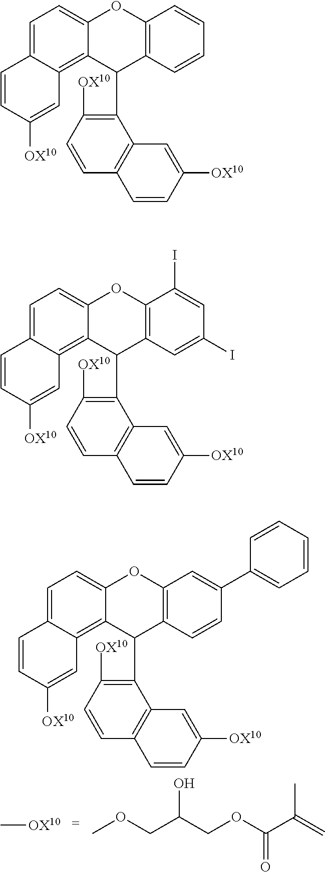

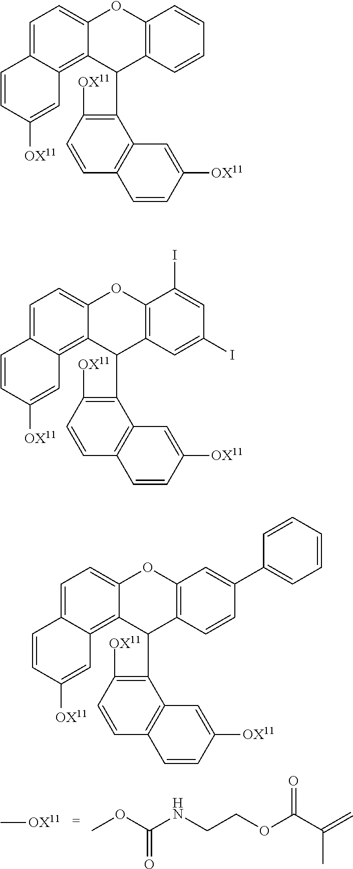

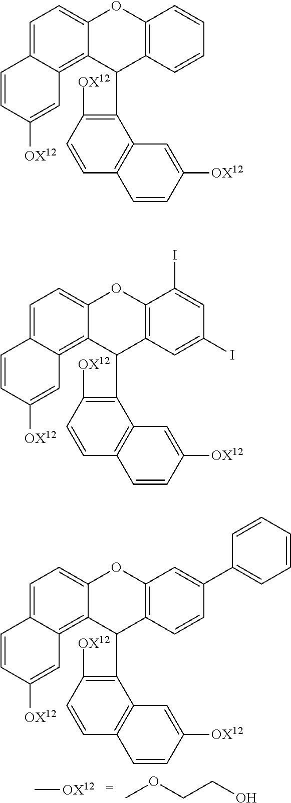

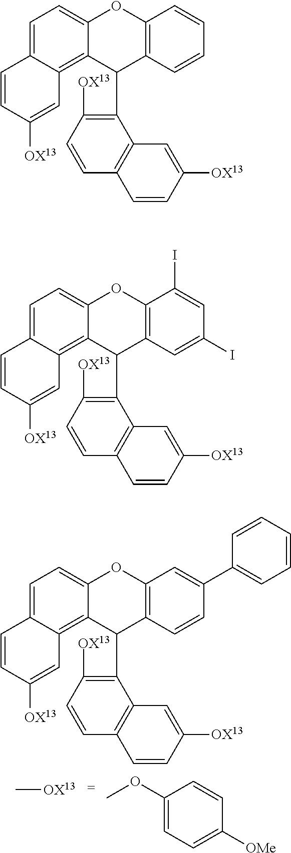

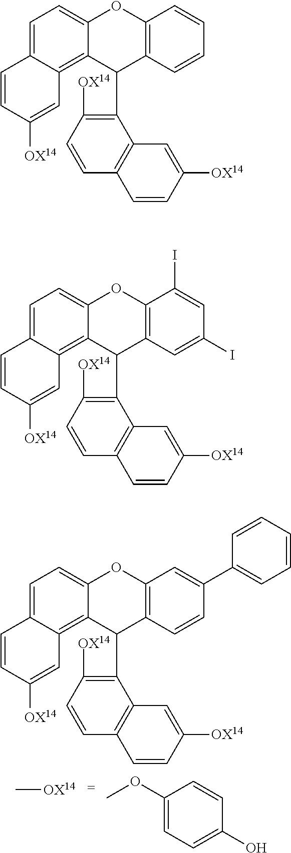

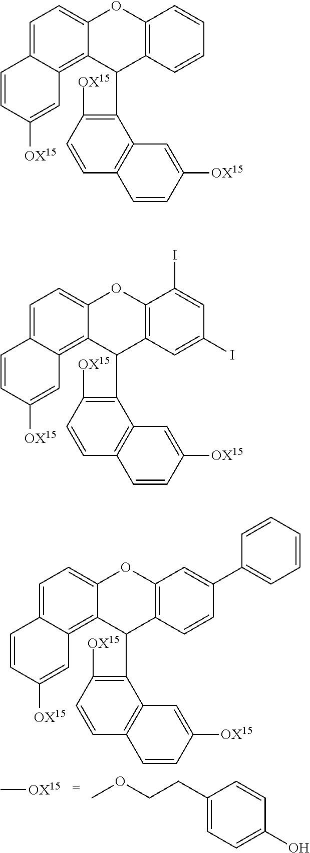

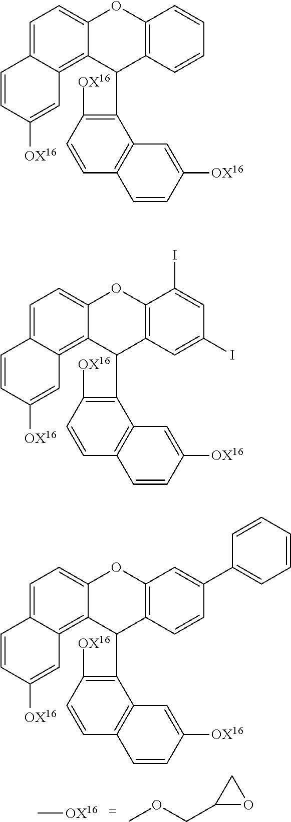

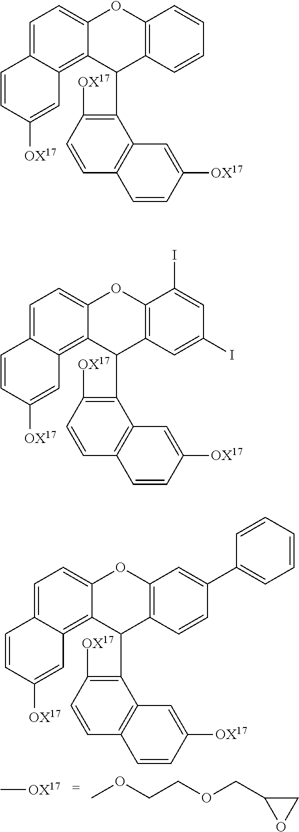

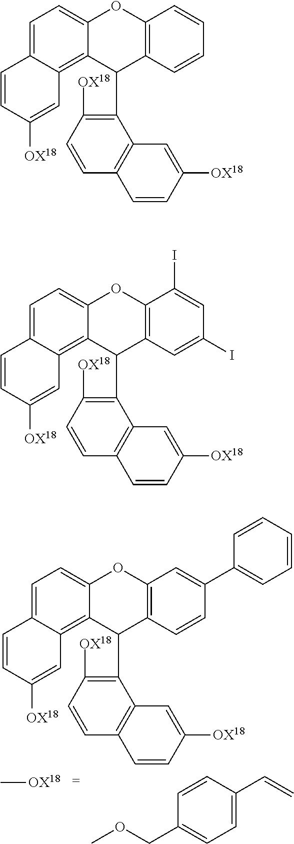

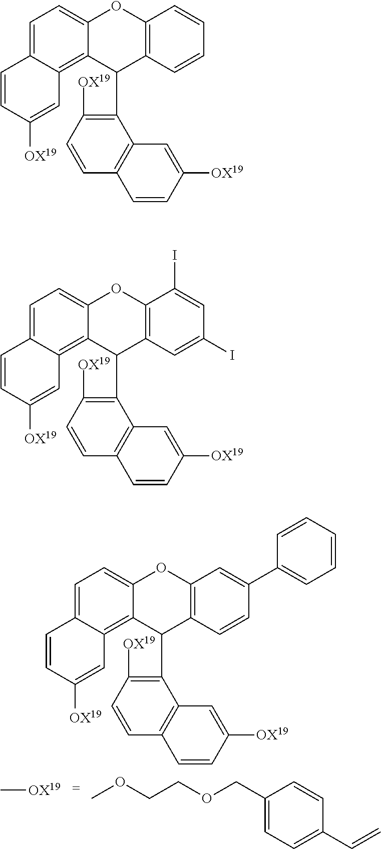

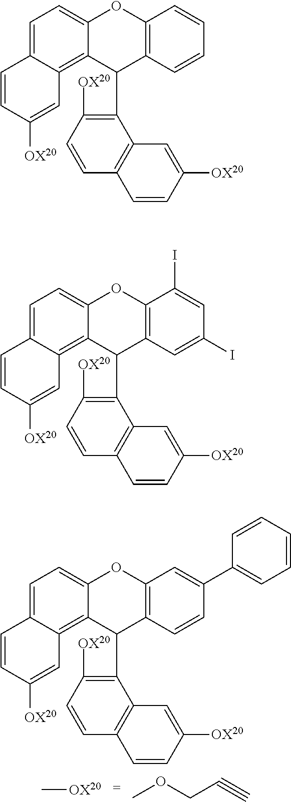

### [Method for Producing Compound]

For the reaction between the aromatic compound represented by the formula (1-1) and the aromatic aldehyde represented by the formula (2-1), publicly known approaches can be arbitrarily applied, and the reaction approach therefor is not particularly limited. A method for producing the compound of the present embodiment comprises a step of subjecting the aromatic compound represented by the formula (1-1) and the formula (2-1) to a condensation reaction, thereby obtaining the skeleton represented by the formula (3-1). In the method for producing the compound of the present embodiment, for example, it is preferable to carry out the condensation reaction at normal pressure and in the presence of an acid catalyst. Alternatively, the reaction may be carried out under increased pressure, if required.

The acid catalyst to be used in the above reaction can be arbitrarily selected for use from publicly known acid catalysts and is not particularly limited. As for the acid catalyst, inorganic acids and organic acids are widely known, and examples thereof include, for example, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; solid acids such as tungstosilicic acid, tungstophosphoric acid, silicomolybdic acid, and phosphomolybdic acid; and the like.

Among the above, organic acids and solid acids are preferable from the viewpoint of production efficiency, and hydrochloric acid or sulfuric acid is more preferably used from the viewpoint of easy availability, handleability, and the like. The acid catalysts can be used alone as one kind, or can be used in combination of two or more kinds.

Also, although the amount of the acid catalyst to be used can be arbitrarily set according to, for example, the kind of the raw materials to be used and the catalyst to be used and moreover the reaction conditions and is not particularly limited, it is preferably 0.01 to 100 parts by mass based on 100 parts by mass of the reaction raw materials.

Upon the above reaction, a reaction solvent may be used. The reaction solvent is not particularly limited as long as it does not hinder the reaction, and it can be arbitrarily selected for use from among publicly known solvents. Examples of the reaction solvent include, for example, water, methanol, ethanol, propanol, butanol, tetrahydrofuran, dioxane, ethylene glycol dimethyl ether, ethylene glycol diethyl ether, and the like. The reaction solvents can be used alone as one kind, or can be used in combination of two or more kinds as a mixed solvent.

Also, although the amount of these reaction solvents to be used can be arbitrarily set according to, for example, the kind of the raw materials to be used and the catalyst to be used and moreover the reaction conditions and is not particularly limited, it is preferably in the range of 0 to 2000 parts by mass based on 100 parts by mass of the reaction raw materials.

The reaction temperature in the above reaction can be arbitrarily selected according to the reactivity of the reaction raw materials and is not particularly limited, but is normally within the range of 10 to 200° C. From the viewpoint of efficiently obtaining the compound of the present embodiment, the reaction temperature is preferably 60 to 200° C.

Note that the reaction method can be arbitrarily selected for use from publicly known approaches and is not particularly limited, and mention may be made of a method in which the aromatic compound represented by the formula (1-1), the aromatic aldehyde represented by the formula (2-1), and the catalyst are charged in one portion, a method in which the aromatic compound represented by the formula (1-1) and the aromatic aldehyde represented by the formula (2-1) are dropped into a system in which the catalyst is present, and the like. After the condensation reaction terminates, isolation of the obtained compound can be carried out according to a conventional method, and is not particularly limited. For example, by adopting a commonly used approach in which the temperature inside the reaction vessel is elevated to 130 to 230° C. in order to remove unreacted raw materials, catalyst, and the like present in the system, and volatile portions are removed at about 1 to 50 mmHg, the compound that is the target compound can be obtained.

Examples of the preferable reaction conditions include conditions, under which 1.0 mol to an excess amount of the aromatic compound represented by the formula (1-1) and 0.001 to 1 mol of the acid catalyst are used based on 1 mol of the aromatic aldehyde represented by the formula (2-1), allowing them to react at 50 to 150° C. at normal pressure for about 20 minutes to 100 hours.

The target compound can be isolated by a publicly known method after the reaction terminates. The compound represented by the formula (3-1), which is the target compound, can be obtained by, for example, concentrating the reaction solution, precipitating the reaction product by the addition of pure water, cooling the reaction solution to room temperature, then separating the precipitates by filtration, filtering and drying the obtained solid matter, then separating and purifying the solid matter from by-products by column chromatography using silica gel or the like, and distilling off the solvent, followed by filtration and drying.

### Resin Obtained with Compound of the Present Embodiment as Monomer

The compound of the present embodiment can be used as is, as a film forming composition for lithography. Also, a resin obtained with the compound of the present embodiment as a monomer can be used as a film forming composition for lithography. One aspect of the present embodiment is a resin, and that resin is a resin having a unit structure derived from the compound of the present embodiment. The resin of the present embodiment is a resin obtained by allowing the compound of the present embodiment to react with a crosslinking compound.

Examples of the resin obtained with the compound of the present embodiment as a monomer include, for example, a resin having a structure represented by the following formula (4). The composition of the present embodiment may contain a resin having a structure represented by the formula (4).

[symbol]

L₂-M [symbol] (4)

(In the formula (4), L₂ is a divalent group having 1 to 60 carbon atoms and M is a unit structure derived from the compound of the present embodiment.)

### Method for Producing Resin Obtained with Compound of the Present Embodiment as Monomer

The resin of the present embodiment is obtained by allowing the compound of the present embodiment to react with a crosslinking compound.

The crosslinking compound is not particularly limited as long as it is capable of oligomerizing or polymerizing the compound of the present embodiment, and publicly known crosslinking compounds can be used. Examples of the crosslinking compound include, for example, aldehydes, ketones, carboxylic acids, carboxylic acid halides, halogen containing compounds, amino compounds, imino compounds, isocyanates, unsaturated hydrocarbon group containing compounds, and the like. These crosslinking compounds may be used alone as one kind, or may be used in combination of two or more kinds.

Specific examples of the resin obtained with the compound of the present embodiment as a monomer include, for example, a resin that is made novolac by, for example, subjecting the compound of the present embodiment to a condensation reaction with an aldehyde and/or ketone that is a crosslinking compound.

Examples of the aldehyde to be used upon making the compound of the present embodiment novolac include, but are not particularly limited to, for example, formaldehyde, trioxane, paraformaldehyde, benzaldehyde, acetaldehyde, propylaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, biphenylaldehyde, naphthaldehyde, anthracenecarbaldehyde, phenanthrenecarbaldehyde, pyrenecarbaldehyde, furfural, and the like.

Examples of the ketone to be used upon making the compound of the present embodiment novolac include, but are not particularly limited to, acetone, 2-butanone, 2-heptanone, 3-heptanone, 4-heptanone, cyclopentanone (CPN), cyclohexanone (CHN), acetophenone, benzophenone, phenyl naphthyl ketone, and the like.

Among the above, formaldehyde is preferable.

Note that these aldehydes and/or ketones can be used alone as one kind, or can be used in combination of two or more kinds. In addition, although the amount of the above aldehyde and/or ketone to be used is not particularly limited, it is preferably 0.2 to 5 mol and is more preferably 0.5 to 2 mol based on 1 mol of the compound of the present embodiment.

A catalyst can also be used in the condensation reaction between the compound of the present embodiment and the aldehyde and/or ketone. The acid catalyst to be used here is not particularly limited, and can be arbitrarily selected for use from publicly known acid catalysts.

As for the acid catalyst, inorganic acids and organic acids are widely known, and examples thereof include, for example, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; solid acids such as tungstosilicic acid, tungstophosphoric acid, silicomolybdic acid, and phosphomolybdic acid; and the like.

Among the above, organic acids and solid acids are preferable from the viewpoint of production efficiency, and hydrochloric acid or sulfuric acid is more preferably used from the viewpoint of production such as easy availability and handleability. The acid catalysts can be used alone as one kind or can be used in combination of two or more kinds.

Also, although the amount of the acid catalyst to be used can be arbitrarily set according to, for example, the kind of the raw materials to be used and the catalyst to be used and moreover the reaction conditions and is not particularly limited, it is preferably 0.01 to 100 parts by mass based on 100 parts by mass of the reaction raw materials. However, the aldehyde is not necessarily needed in the case of a copolymerization reaction with a compound having a non-conjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, and limonene.

A reaction solvent can also be used in the condensation reaction between the compound of the present embodiment and the aldehyde and/or ketone. The reaction solvent in this polycondensation is not particularly limited, and can be arbitrarily selected for use from among publicly known solvents. Examples of the reaction solvent include, for example, water, methanol, ethanol, propanol, butanol, tetrahydrofuran, dioxane, and the like. The reaction solvents can be used alone as one kind or can be used in combination of two or more kinds as a mixed solvent.

In addition, although the amount of these reaction solvents to be used can be arbitrarily set according to, for example, the kind of the raw materials to be used and the catalyst to be used and moreover the reaction conditions and is not particularly limited, it is preferably in the range of 0 to 2000 parts by mass based on 100 parts by mass of the reaction raw materials.

The reaction temperature can be arbitrarily selected according to the reactivity of the reaction raw materials and is not particularly limited, but is normally within the range of 10 to 200° C.

Note that the reaction method can be arbitrarily selected for use from publicly known approaches and is not particularly limited, and mention may be made of a method in which the compound of the present embodiment, the aldehyde and/or ketone, and the catalyst are charged in one portion, and a method in which the compound of the present embodiment and the aldehyde and/or ketone are dropped into a system in which the catalyst is present.

After the polymerization reaction terminates, isolation of the obtained resin can be carried out according to a conventional method, and is not particularly limited. For example, by adopting a commonly used approach in which the temperature inside the reaction vessel is elevated to 130 to 230° C. in order to remove unreacted raw materials, catalyst, and the like present in the system, and volatile portions are removed at about 1 to 50 mmHg, the resin that is the target compound can be obtained.

The resin having a structure represented by the formula (4) may be a homopolymer of the compound of the present embodiment, or may be a copolymer with a further phenolic compound.

The further phenolic compound is not particularly limited as long as it is copolymerizable, and examples thereof include, for example, phenol, cresol, dimethylphenol, trimethylphenol, butylphenol, phenylphenol, diphenylphenol, naphthylphenol, resorcinol, methylresorcinol, catechol, butylcatechol, methoxyphenol, propylphenol, pyrogallol, thymol, and the like.

In addition, the resin having a structure represented by the formula (4) may be a resin copolymerized with a polymerizable monomer other than the further phenolic compound mentioned above. Such a monomer is not particularly limited, and examples thereof include, for example, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornene, pinene, limonene, and the like. Note that the resin having a structure represented by the formula (4) may be a copolymer of two or more components (for example, a binary to quaternary system) composed of the compound of the present embodiment and the phenol mentioned above, may be a copolymer of two or more components (for example, a binary to quaternary system) composed of the compound of the present embodiment and the monomer mentioned above, or may be a copolymer of three or more components (for example, a tertiary to quaternary system) composed of the compound of the present embodiment, the phenolic compound mentioned above, and the monomer mentioned above.

The molecular weight of the resin having a structure represented by the formula (4) is not particularly limited, and the weight average molecular weight (Mw) in terms of polystyrene is preferably 500 to 30,000 and is more preferably 750 to 20,000. Also, from the viewpoint of enhancing crosslinking efficiency while suppressing volatile components during baking, it is preferable that the resin having a structure represented by the formula (4) have a dispersity (weight average molecular weight Mw/number average molecular weight Mn) within the range of 1.2 to 7.

The above Mn can be determined by a method described in Examples mentioned later.

The resin having a structure represented by the formula (4) preferably has high solubility in a solvent from the viewpoint of easier application to a wet process and the like. More specifically, in the case of using 1-methoxy-2-propanol (PGME) and/or propylene glycol monomethyl ether acetate (PGMEA) as a solvent, it is preferable that the compound and/or resin have a solubility of 10% by mass or more in the solvent. Here, the solubility in PGME and/or PGMEA is defined as “mass of the resin/(mass of the resin+mass of the solvent)×100 (% by mass)”. For example, when 10 g of the resin is dissolved in 90 g of PGMEA, the solubility of the resin in PGMEA is “10% by mass or more”; and when 10 g of the resin is not dissolved in 90 g of PGMEA, the solubility is “less than 10% by mass”.

### [Purification Method]

The compound of the present embodiment and the resin of the present embodiment can be purified by washing with an acidic aqueous solution. One aspect of the present embodiment is a method for purifying the compound of the present embodiment or resin of the present embodiment (they may also be referred to as a film forming material for lithography), and the purification method comprises an extraction step in which extraction is carried out by bringing a solution containing the compound or resin, and an organic solvent that does not inadvertently mix with water into contact with an acidic aqueous solution.

The purification method of the present embodiment specifically comprises a step in which the film forming material for lithography is dissolved in an organic solvent that does not inadvertently mix with water to obtain an organic phase, the organic phase is brought into contact with an acidic aqueous solution to carry out an extraction treatment (a first extraction step), thereby transferring metals contained in the organic phase containing the film forming material for lithography and the organic solvent to an aqueous phase, and then, the organic phase and the aqueous phase are separated. According to the purification, the contents of various metals in the film forming material for lithography of the present invention can be reduced remarkably.

The organic solvent that does not inadvertently mix with water is not particularly limited, but is preferably an organic solvent that is safely applicable to semiconductor manufacturing processes. Normally, the amount of the organic solvent to be used is approximately 1 to 100 times by mass relative to the compound to be used.

Specific examples of the organic solvent to be used include, for example, those described in International Publication No. WO 2015/080240. Among the above, toluene, 2-heptanone, cyclohexanone, cyclopentanone, methyl isobutyl ketone, propylene glycol monomethyl ether acetate, ethyl acetate, and the like are preferable, and cyclohexanone and propylene glycol monomethyl ether acetate are particularly preferable. These organic solvents can be each used alone, or can also be used as a mixture of two or more kinds.

The above acidic aqueous solution is appropriately selected from aqueous solutions in which generally known organic or inorganic compounds are dissolved in water. For example, examples thereof include those described in International Publication No. WO 2015/080240. These acidic aqueous solutions can be each used alone, or can also be used as a combination of two or more kinds. Examples of the acidic aqueous solution may include, for example, an aqueous mineral acid solution and an aqueous organic acid solution. Examples of the aqueous mineral acid solution may include, for example, an aqueous solution comprising one or more selected from the group consisting of hydrochloric acid, sulfuric acid, nitric acid, and phosphoric acid. Examples of the aqueous organic acid solution may include, for example, an aqueous solution comprising one or more selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid. Moreover, as the acidic aqueous solution, aqueous solutions of sulfuric acid, nitric acid, and a carboxylic acid such as acetic acid, oxalic acid, tartaric acid, and citric acid are preferable, aqueous solutions of sulfuric acid, oxalic acid, tartaric acid, and citric acid are still more preferable, and an aqueous solution of oxalic acid is particularly preferable. It is considered that a polyvalent carboxylic acid such as oxalic acid, tartaric acid, and citric acid coordinates with metal ions and provides a chelating effect, and thus is capable of removing more metals. In addition, as the water used here, water, the metal content of which is small, such as ion exchanged water, is preferable according to the purpose of the present invention.

The pH of the acidic aqueous solution is not particularly limited, but when the acidity of the aqueous solution is too high, it may have a negative influence on the used compound or resin, which is not preferable. Normally, the pH range is about 0 to 5, and is more preferably about pH 0 to 3.

The amount of the acidic aqueous solution to be used is not particularly limited, but when the amount is too small, it is required to increase the number of extraction treatments for removing metals, and on the other hand, when the amount of the aqueous solution is too large, the entire fluid volume becomes large, which may cause operational problems. Normally, the amount of the aqueous solution to be used is 10 to 200 parts by mass and is preferably 20 to 100 parts by mass relative to the solution of the compound.

By bringing the acidic aqueous solution into contact with a solution (B) containing the compound and the organic solvent that does not inadvertently mix with water, metals can be extracted.

The temperature at which the above extraction treatment is carried out is generally in the range of 20 to 90° C., and preferably 30 to 80° C. The extraction operation is carried out, for example, by thoroughly mixing the solution (B) and the acidic aqueous solution by stirring or the like and then leaving the obtained mixed solution to stand still. Thereby, metals contained in the solution containing the used compound and the organic solvent are transferred to the aqueous phase. Also, by this operation, the acidity of the solution is lowered, and the deterioration of the used compound can be suppressed.

After the extraction treatment, the mixed solution is separated into a solution phase containing the used compound and the organic solvent and an aqueous phase, and the solution containing the organic solvent is recovered by decantation or the like. The time for leaving the mixed solution to stand still is not particularly limited, but when the time for leaving the mixed solution to stand still is too short, separation of the solution phase containing the organic solvent and the aqueous phase becomes poor, which is not preferable. Normally, the time for leaving the mixed solution to stand still is 1 minute or longer, more preferably 10 minutes or longer, and still more preferably 30 minutes or longer. In addition, while the extraction treatment may be carried out only once, it is also effective to repeat mixing, leaving-to-stand-still, and separating operations multiple times.

When such an extraction treatment is carried out using the acidic aqueous solution, after the treatment, it is preferable to further subjecting the recovered organic phase that has been extracted from the aqueous solution and contains the organic solvent to an extraction treatment with water (a second extraction step). The extraction operation is carried out by thoroughly mixing the organic phase and water by stirring or the like and then leaving the obtained mixed solution to stand still. The resultant mixed solution is separated into a solution phase containing the compound and the organic solvent and an aqueous phase, and thus the solution phase is recovered by decantation or the like. In addition, water used here is preferably water, the metal content of which is small, such as ion exchanged water, according to the purpose of the present invention. While the extraction treatment may be carried out only once, it is also effective to repeat mixing, leaving-to-stand-still, and separating operations multiple times. The proportions of both used in the extraction treatment and the temperature, time, and other conditions are not particularly limited, and may be the same as those of the previous contact treatment with the acidic aqueous solution.

Water that is present in the thus-obtained solution containing the compound and the organic solvent can be easily removed by performing vacuum distillation or a like operation. Also, if required, the concentration of the compound can be regulated to be any concentration by adding an organic solvent.

A method for only obtaining the objective compound from the obtained solution containing the organic solvent can be carried out through a publicly known method such as reduced-pressure removal, separation by reprecipitation, and a combination thereof. Publicly known treatments such as concentration operation, filtration operation, centrifugation operation, and drying operation can be carried out if required.

### [Composition]

The composition of the present embodiment comprises the compound of the present embodiment and/or the resin of the present embodiment, and may also comprise other components such as a base material (A), a solvent (S), an acid generating agent (C), a crosslinking agent (G), and an acid diffusion controlling agent (E), if required. Hereinafter, each of these components will be described.

### (Base Material (A))

A “base material (A)” in the present embodiment is a compound (including a resin) other than the compound of the present embodiment or the resin of the present embodiment, and means a base material applied as a resist for g-ray, i-ray, KrF excimer laser (248 nm), ArF excimer laser (193 nm), extreme ultraviolet (EUV) lithography (13.5 nm) or electron beam (EB) (for example, a base material for lithography or a base material for resist).

The base material (A) in the present embodiment is not particularly limited, and examples thereof include, for example, a phenol novolac resin, a cresol novolac resin, a hydroxystyrene resin, a (meth)acrylic resin, a hydroxystyrene-(meth)acrylic copolymer, a cycloolefin-maleic anhydride copolymer, a cycloolefin, a vinyl ether-maleic anhydride copolymer, and an inorganic resist material having a metallic element such as titanium, tin, hafnium, and zirconium, and a derivative thereof.

Among the above, from the viewpoint of the shape of a resist pattern to be obtained, preferable are a phenol novolac resin, a cresol novolac resin, a hydroxystyrene resin, a (meth)acrylic resin, a hydroxystyrene-(meth)acrylic copolymer, and an inorganic resist material having a metallic element such as titanium, tin, hafnium, and zirconium, and a derivative thereof.

Examples of the above derivative include, but are not particularly limited to, a derivative to which a dissociation group is introduced, a derivative to which a crosslinkable group is introduced, and the like. The above derivative to which a dissociation group or a crosslinkable group is introduced can exhibit dissociation reaction or crosslinking reaction through the effect of light, acid, or the like. As the dissociation group and the crosslinkable group, the same groups as the dissociation groups and the crosslinkable groups included in the aromatic compound represented by the formula (1-1) and the aromatic aldehyde represented by the formula (2-1) in the present embodiment can be exemplified.

In the present embodiment, the weight average molecular weight of the base material (A) is preferably 200 to 4990, more preferably 200 to 2990, and still more preferably 200 to 1490 from the viewpoints of reducing defects in a film to be formed by using the composition and of a good pattern shape. As the above weight average molecular weight, a value obtained by measuring the weight average molecular weight in terms of polystyrene, using GPC, can be used.

### [Solvent (S)]

A solvent in the present embodiment is not particularly limited as long as it can at least dissolve the compound of the present embodiment, and a publicly known solvent can be arbitrarily used. Examples of the solvent may include, for example, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate (PGMEA), propylene glycol monoethyl ether acetate, propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; ester lactates such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as acetone, 2-butanone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; lactones such as γ-lactone; and the like. The solvent used in the present embodiment is preferably a safe solvent, more preferably at least one selected from PGMEA, PGME, CHN, CPN, 2-heptanone, anisole, butyl acetate, and ethyl lactate, and still more preferably at least one selected from PGMEA, PGME, CHN, CPN, and ethyl lactate.

In the present embodiment, the amount of the solid components and the amount of the solvent are not particularly limited, but preferably the solid components is 1 to 80% by mass and the solvent is 20 to 99% by mass, more preferably the solid components is 1 to 50% by mass and the solvent is 50 to 99% by mass, still more preferably the solid components is 2 to 40% by mass and the solvent is 60 to 98% by mass, and yet even more preferably the solid components is 2 to 10% by mass and the solvent is 90 to 98% by mass, based on the total mass of the amount of the solid components and the solvent.

### [Acid Generating Agent (C)]

The composition of the present embodiment preferably comprises one or more acid generating agents (C) generating an acid directly or indirectly by irradiation of any radiation selected from visible light, ultraviolet, excimer laser, electron beam, extreme ultraviolet (EUV), X-ray, and ion beam. The acid generating agent (C) is not particularly limited, and, for example, an acid generating agent described in International Publication No. WO 2013/024778 can be used. The acid generating agent (C) can be used alone, or can be used in combination of two or more kinds.

The amount of the acid generating agent (C) to be used is preferably 0.001 to 49% by mass of the total mass of the solid components, more preferably 1 to 40% by mass, still more preferably 3 to 30% by mass, and yet even more preferably 10 to 25% by mass. By using the acid generating agent (C) within the above range, there is a tendency that a pattern profile with high sensitivity and low edge roughness is obtained. In the present embodiment, the acid generation method is not particularly limited as long as an acid is generated in the system. By using excimer laser instead of ultraviolet such as g-ray and i-ray, finer processing is possible, and also by using electron beam, extreme ultraviolet, X-ray, or ion beam as a high energy ray, further finer processing is possible.

### [Acid Diffusion Controlling Agent (E)]

In the present embodiment, the composition may contain an acid diffusion controlling agent (E), which has a function of controlling diffusion of an acid generated from the acid generating agent by radiation irradiation in a resist film to inhibit any unpreferable chemical reaction in an unexposed region or the like. By using the acid diffusion controlling agent (E), there is a tendency that the storage stability of the composition of the present embodiment can be improved. Also, by using the acid diffusion controlling agent (E), there is a tendency that, not only the resolution of a film formed by using the composition of the present embodiment can be improved, but the line width change of a resist pattern due to variation in the post exposure delay time before radiation irradiation and the post exposure delay time after radiation irradiation can also be inhibited, making the composition excellent in process stability. Examples of the acid diffusion controlling agent (E) include, but are not particularly limited to, a radiation degradable basic compound such as a nitrogen atom containing basic compound, a basic sulfonium compound, and a basic iodonium compound.

The acid diffusion controlling agent (E) is not particularly limited, and, for example, an acid diffusion controlling agent described in International Publication No. WO 2013/024778 can be used. The acid diffusion controlling agent (E) can be used alone, or can be used in combination of two or more kinds.

The content of the acid diffusion controlling agent (E) is preferably 0.001 to 49% by mass of the total mass of the solid components, more preferably 0.01 to 10% by mass, still more preferably 0.01 to 5% by mass, and particularly preferably 0.01 to 3% by mass. When the content of the acid diffusion controlling agent (E) is within the above range, there is a tendency that a decrease in resolution, and deterioration of the pattern shape and the dimension fidelity or the like can be prevented. Moreover, even though the post exposure delay time from electron beam irradiation to heating after radiation irradiation becomes longer, deterioration of the shape of the pattern upper layer portion can be suppressed. Also, when the content is 10% by mass or less, there is a tendency that a decrease in sensitivity, and developability of the unexposed portion or the like can be prevented. Also, by using such an acid diffusion controlling agent, there is a tendency that the storage stability of a resist composition is improved, also along with improvement of the resolution, the line width change of a resist pattern due to variation in the post exposure delay time before radiation irradiation and the post exposure delay time after radiation irradiation can be inhibited, making the composition excellent in process stability.

### [Crosslinking Agent (G)]

A crosslinking agent (G) of the present embodiment is not particularly limited, and, for example, a crosslinking agent described in International Publication No. WO 2013/024778 can be used. The crosslinking agent (G) can be used alone, or can be used in combination of two or more kinds.

### [Further Component (F)]

To the composition of the present embodiment, if required, as a further component (F), one kind or two or more kinds of various additive agents such as a dissolution promoting agent, a dissolution controlling agent, a sensitizing agent, a surfactant, and an organic carboxylic acid or an oxo acid of phosphorus or derivative thereof can be added.

### (Dissolution Promoting Agent)

The dissolution promoting agent is a component having a function of, when the solubility of solid components is too low, increasing the solubility of the solid components in a developing solution to moderately increase the dissolution rate of that compound upon developing. As the above dissolution promoting agent, those having a low molecular weight are preferable, and examples thereof can include a phenolic compound having a low molecular weight. Examples of the phenolic compound having a low molecular weight can include a bisphenol, a tris(hydroxyphenyl)methane, and the like. These dissolution promoting agents can be used alone, or can be used as a mixture of two or more kinds.

The content of the dissolution promoting agent, which is arbitrarily adjusted according to the kind of the above solid components to be used, is preferably 0 to 49% by mass of the total mass of the solid components, more preferably 0 to 5% by mass, still more preferably 0 to 1% by mass, and yet even more preferably 0% by mass.

### (Dissolution Controlling Agent)

The dissolution controlling agent is a component having a function of, when the solubility of solid components is too high, controlling the solubility of the solid components in a developing solution to moderately decrease the dissolution rate upon developing. As such a dissolution controlling agent, the one which does not chemically change in steps such as calcination of resist coating, radiation irradiation, and development is preferable.

The dissolution controlling agent is not particularly limited, and examples thereof can include an aromatic hydrocarbon such as phenanthrene, anthracene, and acenaphthene; a ketone such as acetophenone, benzophenone, and phenyl naphthyl ketone; and a sulfone such as methyl phenyl sulfone, diphenyl sulfone, and dinaphthyl sulfone. These dissolution controlling agents can be used alone, or can be used in combination of two or more kinds.

The content of the dissolution controlling agent, which is arbitrarily adjusted according to the kind of the above compound to be used, is preferably 0 to 49% by mass of the total mass of the solid components, more preferably 0 to 5% by mass, still more preferably 0 to 1% by mass, and yet even more preferably 0% by mass.

### (Sensitizing Agent)

The sensitizing agent is a component having a function of absorbing irradiated radiation energy, transmitting the energy to the acid generating agent (C), and thereby increasing the acid production amount, and improving the apparent sensitivity of a resist. Examples of such a sensitizing agent can include, but are not particularly limited to, a benzophenone, a biacetyl, a pyrene, a phenothiazine, and a fluorene. These sensitizing agents can be used alone, or can be used in combination of two or more kinds.

The content of the sensitizing agent, which is arbitrarily adjusted according to the kind of the above compound to be used, is preferably 0 to 49% by mass of the total mass of the solid components, more preferably 0 to 5% by mass, still more preferably 0 to 1% by mass, and yet even more preferably 0% by mass.

### (Surfactant)

The surfactant is a component having a function of improving coatability and striation of the composition of the present embodiment, and developability of a resist or the like. The surfactant may be any of anionic, cationic, nonionic, and amphoteric surfactants. Preferable examples of the surfactant include a nonionic surfactant. The nonionic surfactant has a good affinity with a solvent to be used in production of the composition of the present embodiment, and can further enhance the effects of the composition of the present embodiment. Examples of the nonionic surfactant include, but are not particularly limited to, a polyoxyethylene higher alkyl ether, a polyoxyethylene higher alkyl phenyl ether, a higher fatty acid diester of polyethylene glycol, and the like. Examples of commercially available products of these surfactants can include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), Polyflow (manufactured by Kyoeisha Chemical Co., Ltd.), and the like.

The content of the surfactant, which is arbitrarily adjusted according to the kind of the above solid components to be used, is preferably 0 to 49% by mass of the total mass of the solid components, more preferably 0 to 5% by mass, still more preferably 0 to 1% by mass, and yet even more preferably 0% by mass.

### (Organic Carboxylic Acid or Oxo Acid of Phosphorus or Derivative Thereof)

For the purpose of prevention of sensitivity deterioration or improvement of a resist pattern shape and post exposure delay stability or the like, and as an additional optional component, the composition of the present embodiment can contain an organic carboxylic acid or an oxo acid of phosphorus or derivative thereof. The organic carboxylic acid or the oxo acid of phosphorus or derivative thereof can be used in combination with the acid diffusion controlling agent, or may be used alone. The organic carboxylic acid is, for example, suitably malonic acid, citric acid, malic acid, succinic acid, benzoic acid, salicylic acid, or the like. Examples of the oxo acid of phosphorus or derivative thereof include phosphoric acid or derivative thereof such as ester including phosphoric acid, di-n-butyl ester phosphate, and diphenyl ester phosphate; phosphonic acid or derivative thereof such as ester including phosphonic acid, dimethyl ester phosphonate, di-n-butyl ester phosphonate, phenylphosphonic acid, diphenyl ester phosphonate, and dibenzyl ester phosphonate; and phosphinic acid and derivative thereof such as ester including phosphinic acid and phenylphosphinic acid. Among the above, phosphonic acid is particularly preferable.

The organic carboxylic acid or the oxo acid of phosphorus or derivative thereof can be used alone, or can be used in combination of two or more kinds. The content of the organic carboxylic acid or the oxo acid of phosphorus or derivative thereof, which is arbitrarily adjusted according to the kind of the above compound to be used, is preferably 0 to 49% by mass of the total mass of the solid components, more preferably 0 to 5% by mass, still more preferably 0 to 1% by mass, and yet even more preferably 0% by mass.

### (Additional Additive Agent)

Furthermore, the composition of the present embodiment can contain one kind or two or more kinds of additive agents other than the components mentioned above, if required. Examples of such an additive agent include a dye, a pigment, and an adhesion aid. For example, when the composition contains a dye or a pigment, a latent image of the exposed portion is visualized and influence of halation upon exposure can be alleviated, which is preferable. Also, when the composition contains an adhesion aid, adhesiveness to a substrate can be improved, which is preferable. Furthermore, examples of the additional additive agent can include a halation preventing agent, a storage stabilizing agent, a defoaming agent, and a shape improving agent. Specific examples thereof can include 4-hydroxy-4′-methylchalkone.

In the composition of the present embodiment, the total content of the optional component (F) can be 0 to 99% by mass of the total mass of the solid components, and is preferably 0 to 49% by mass, more preferably 0 to 10% by mass, still more preferably 0 to 5% by mass, further preferably 0 to 1% by mass, and yet even more preferably 0% by mass.

The composition of the present embodiment can be used in film formation for lithography, film formation for resist, resist underlayer film formation, and optical component formation.

### [Composition for Film Formation for Lithography and Composition for Film Formation For Resist]

A composition for film formation for lithography and composition for film formation for resist of the present embodiment can form a desired cured film by applying them on a base material, subsequently heating them to evaporate the solvent if necessary, and then heating or photoirradiating them. A method for applying the composition for film formation for lithography and composition for film formation for resist of the present embodiment is arbitrary, and a method such as spin coating, dipping, flow coating, inkjet coating, spraying, bar coating, gravure coating, slit coating, roll coating, transfer printing, brush coating, blade coating, and air knife coating can be arbitrarily employed.

The temperature at which the film is heated is not particularly limited according to the purpose of evaporating the solvent, and the heating can be carried out at, for example, 40 to 400° C. A method for heating is not particularly limited, and for example, the solvent may be evaporated under an appropriate atmosphere such as atmospheric air, an inert gas including nitrogen, and vacuum by using a hot plate or an oven. For the heating temperature and heating time, it is only required to select conditions suitable for a processing step for an electronic device that is aimed at and to select heating conditions by which physical property values of the obtained film satisfy requirements of the electronic device. Conditions for photoirradiation are not particularly limited, either, and it is only required to employ appropriate irradiation energy and irradiation time depending on a film forming material for lithography and film formation for resist to be used.

### [Method for Forming Resist Underlayer Film and Resist Pattern]

The composition of the present embodiment is used in formation of a resist underlayer film and a resist pattern.

A method for forming a resist pattern of the present embodiment comprises: a photoresist layer formation step of forming a photoresist layer on a substrate using the composition for film formation for lithography or composition for film formation for resist of the present embodiment; and a development step of irradiating a predetermined region of the photoresist layer formed through the photoresist layer formation step with radiation for development.

Also, the method for forming a resist pattern of the present embodiment comprises: an underlayer film formation step of forming an underlayer film on a substrate using the composition of the present embodiment; a photoresist layer formation step of forming at least one photoresist layer on the underlayer film formed through the underlayer film formation step; and a step of irradiating a predetermined region of the photoresist layer formed through the photoresist layer formation step with radiation for development.

In order to form a resist pattern and an underlayer film from the composition of the present embodiment, specifically, a substrate such as a silicon wafer, metal, plastic, glass, or ceramic is coated with the composition by an appropriate coating means such as a spin coater, a dip coater, or a roller coater, thereby forming a resist coating; optionally subjected to a heat treatment in advance at a temperature of about 50° C. to 200° C.; and then exposed through a predetermined mask pattern. The thickness of the coating film is, for example, 0.1 to 20 μm, and is preferably about 0.3 to 2 μm. For the exposure, lights with a variety of wavelengths, such as ultraviolet and X-ray, can be used, and as the light source, for example, far ultraviolet such as F2 excimer laser (wavelength of 157 nm), ArF excimer laser (wavelength of 193 nm), or KrF excimer laser (wavelength of 248 nm), extreme ultraviolet (wavelength of 13 n), X-ray, electron beam, or the like can be arbitrarily selected for use. Also, the exposure conditions, such as the amount of exposure, are arbitrarily selected depending on the compounding composition of the above resin and/or compound, the type of each additive agent, and the like.

It is preferable that the resist pattern of the present embodiment is an insulating film pattern.

In the present embodiment, in order to stably form a fine pattern with a high degree of accuracy, it is preferable to carry out a heating treatment at a temperature of 50 to 200° C. for 30 seconds or longer after the exposure. In this case, when the temperature is lower than 50° C., there is a risk that the variation in sensitivity due to the type of substrate may be broadened. Thereafter, by using an alkaline developing solution for development normally under conditions of 10 to 50° C. for 10 to 200 seconds, preferably 20 to 25° C. for 15 to 90 seconds, a predetermined resist pattern is formed.

As the above alkaline developing solution, for example, an alkaline aqueous solution is used in which an alkaline compound such as an alkali metal hydroxide, ammonia water, an alkylamine, an alkanolamine, heterocyclic amine, a tetraalkylammonium hydroxide, corrin, 1,8-diazabicyclo-[5.4.0]-7-undecene, and 1,5-diazabicyclo-[4.3.0]-5-nonene is dissolved to a concentration of normally 1 to 10% by weight, preferably 1 to 3% by weight. In addition, to the developing solution comprising the above alkaline aqueous solution, a water soluble organic solvent or surfactant can also be arbitrarily added.

Moreover, one aspect of the present embodiment is a method for forming a circuit pattern, the method comprising: an underlayer film formation step of forming an underlayer film on a substrate using the composition of the present embodiment; an intermediate layer film formation step of forming an intermediate layer film on the underlayer film formed through the underlayer film formation step; a photoresist layer formation step of forming at least one photoresist layer on the intermediate layer film formed through the intermediate layer film formation step; a resist pattern formation step of irradiating a predetermined region of the photoresist layer formed through the photoresist layer formation step with radiation for development, thereby forming a resist pattern; an intermediate layer film pattern formation step of etching the intermediate layer film with the resist pattern formed through the resist pattern formation step as a mask, thereby forming an intermediate layer film pattern; an underlayer film pattern formation step of etching the underlayer film with the intermediate layer film pattern formed through the intermediate layer film pattern formation step as a mask, thereby forming an underlayer film pattern; and a substrate pattern formation step of etching the substrate with the underlayer film pattern formed through the underlayer film pattern formation step as a mask, thereby forming a pattern on the substrate.

### [Composition for Optical Component Formation]

In addition, since a film obtained from a composition comprising the compound of the present embodiment also has a high refractive index, the compound and composition of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, and the like), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). It can be particularly suitably utilized as an embedded film and a smoothed film on a photodiode, a smoothed film in front of or behind a color filter, a microlens, and a smoothed film and a conformal film on a microlens, all of which are members of a solid state image sensing element, to which high refractive index is demanded.

### EXAMPLES

Hereinafter, the present invention will be described in further detail with reference to Examples and Comparative Examples, but the present invention is not limited by these Examples in any way.

### [Molecular Weight]

The molecular weight of a compound was measured by LC-MS analysis using Acquity UPLC/MALDI-Synapt HDMS manufactured by Waters Corp.

Also, the weight average molecular weight (Mw), number average molecular weight (Mn), and dispersity (Mw/Mn) in terms of polystyrene were determined by gel permeation chromatography (GPC) analysis under the following conditions.

Apparatus: Shodex GPC-101 model (manufactured by Showa Denko K.K.)

Column: KF-80M×3

Eluent: 1 mL/min THF

Temperature: 40° C.

### [Structure of Compound]

The structure of a compound was confirmed by ¹H-NMR measurement using “Advance 600II spectrometer” manufactured by Bruker Corp. under the following conditions.

Frequency: 400 MHz

Solvent: d6-DMSO

Internal standard: TMS

Measurement temperature: 23° C.

### Synthesis Working Example 1-1

### Synthesis of X-27N35IB

In a container (internal capacity: 1 L) equipped with a stirrer, a condenser tube, and a burette, 24 g (150 mmol) of 2,7-dihydroxynaphthalene (a reagent manufactured by Sigma-Aldrich), 25.4 g (71 mmol) of 3,5-diiodosalicylaldehyde (a reagent manufactured by Tokyo Chemical Industry Co., Ltd.), and 200 mL of 1-methoxy-2-propanol were charged, and 1.3 g (14 mmol) of methanesulfonic acid (a reagent manufactured by Kanto Chemical Co., Inc.) was added to prepare a reaction solution. This reaction solution was stirred at 90° C. for 5 hours and allowed to react. After the reaction finished, 1.7 L of pure water was added to the reaction solution, extraction with ethyl acetate was performed, followed by concentration, to obtain a solution. The obtained solution was separated and purified by column chromatography to obtain 9.2 g of the objective compound (X-27N35IB) represented by the following formula (purity: 98.7% and yield: 20%).

As a result of measuring the molecular weight of the obtained compound (X-27N35IB) by the above method, it was 658. Also, since the following peaks were found by performing the ¹H-NMR measurement under the above measurement conditions, the compound was confirmed to have a chemical structure of the following formula (X-27N35IB).

¹H-NMR (d6-DMSO): δ (ppm) 10.4 (1H, —OH), 9.8 (1H, —OH), 9.5 (1H, —OH), 6.7-8.0 (12H, Ph), 6.2 (1H, Methine)

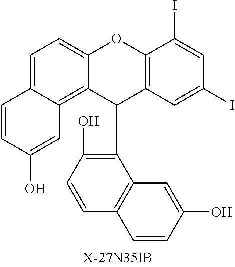 [SMILES: Oc1ccc2ccc(O)c(C3c4cc(I)cc(I)c4Oc4ccc5ccc(O)cc5c43)c2c1]

### Synthesis Working Example 1-2

### Synthesis of X-27N35IB-MeBOC

In a container (internal capacity: 200 mL) equipped with a stirrer, a condenser tube, and a burette, 5.3 g (8.1 mmol) of the compound (X-27N35IB) obtained as described above, 5.4 g (27 mmol) of t-butyl bromoacetate (manufactured by Sigma-Aldrich), and 100 mL of acetone were charged, and 3.8 g (27 mmol) of potassium carbonate (manufactured by Sigma-Aldrich) and 0.8 g of 18-crown-6 were added. The contents were stirred for 3 hours under reflux and allowed to react. Next, after the reaction finished, the reaction solution was concentrated, and the reaction product was precipitated by adding 100 g of pure water to the concentrate, cooled to room temperature, and then filtered to separate solid matter.

The obtained solid matter was dried, and then separated and purified by column chromatography to obtain 1.5 g of the following formula (X-27N35IB-MeBOC).

The following peaks were found by the NMR measurement performed on the obtained compound (X-27N35IB-MeBOC) under the above measurement conditions, and the compound was confirmed to have a chemical structure of the following formula (X-27N35IB-MeBOC).

¹H-NMR (d6-DMSO): δ (ppm) 1.4 (27H, O—C—CH₃), 4.9 (6H, O—CH₂—C), 6.7-8.0 (12H, Ph), 6.2 (1H, Methine)

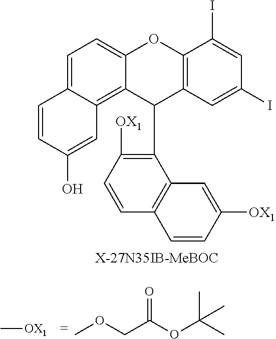

### Synthesis Working Example 1-3

### Synthesis of Resin (R-X-27N35IB)

A four necked flask (internal capacity: 1 L) equipped with a Dimroth condenser tube, a thermometer, and a stirring blade and having a detachable bottom was prepared. To this four necked flask, 25 g (70 mmol) of the compound (X-27N35IB) obtained in Synthesis Working Example 1-1, 21.0 g (280 mmol as formaldehyde) of a 40 mass % aqueous formalin solution (manufactured by Mitsubishi Gas Chemical Company, Inc.), and 0.97 mL of a 98 mass % sulfuric acid (manufactured by Kanto Chemical Co., Inc.) were charged in a nitrogen stream, and the mixture was allowed to react for 7 hours while being refluxed at 100° C. at normal pressure. Subsequently, 180.0 g of orthoxylene (special grade reagent manufactured by Wako Pure Chemical Industries, Ltd.) was added as a diluting solvent to the reaction solution, and the mixture was left to stand still, followed by removal of an aqueous phase as a lower phase. Neutralization and washing with water were further performed, and orthoxylene was distilled off under reduced pressure to obtain 34.1 g of a brown solid resin (R-X-27N35IB).

The molecular weight of the obtained resin (R-X-27N35IB) was Mn: 3970, Mw: 7250, and Mw/Mn: 1.89.

### Synthesis Working Example 1-4

### Synthesis of X-27N35IB-BOC

In a container (internal capacity: 200 mL) equipped with a stirrer, a condenser tube, and a burette, 5.3 g (8.1 mmol) of the compound (X-27N35IB) obtained in Synthesis Working Example 1-1 and 5.2 g (23.8 mmol) of di-t-butyl dicarbonate (manufactured by Sigma-Aldrich) were charged in 100 mL of acetone, 3.29 g (23.8 mmol) of potassium carbonate (manufactured by Sigma-Aldrich) was added thereto, and the contents were allowed to react by being stirred at 20° C. for 6 hours to obtain a reaction solution. Next, the reaction solution was concentrated, and the reaction product was precipitated by the addition of 100 g of pure water to the concentrate, cooled to room temperature, and then filtered to separate solid matter.

The obtained solid matter was filtered and then dried. Subsequently, the solid matter was separated and purified by column chromatography to obtain 0.8 g of the objective compound (X-27N35IB-BOC) represented by the following formula.

The following peaks were found by the NMR measurement performed on the obtained compound under the above measurement conditions, and the compound was confirmed to have a chemical structure of the following formula (X-27N35IB-BOC).

¹H-NMR (d6-DMSO): δ (ppm) 1.4 (27H, O—C—CH₃), 5.3 (1H, C—H), 6.9-7.9 (12H, Ph-H)

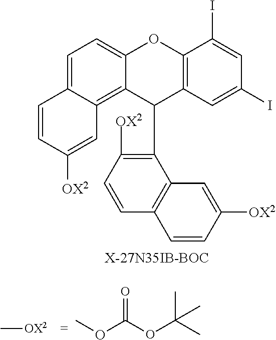

### Synthesis Working Example 1-5

### Synthesis of X-27N35IB-AL

In a container (internal capacity: 500 mL) equipped with a stirrer, a condenser tube, and a burette, 5.3 g (8.1 mmol) of the compound (X-27N35IB) obtained by the method of Synthesis Working Example 1-1, 54 g (39 mmol) of potassium carbonate, and 200 mL of dimethylformamide were charged, 77.6 g (0.64 mol) of allyl bromide was added thereto, and the reaction solution was stirred at 110° C. for 24 hours and allowed to react. Next, the reaction solution was concentrated. The reaction product was precipitated by the addition of 500 g of pure water. After cooling to room temperature, the precipitates were separated by filtration. The obtained solid matter was filtered and then dried. Subsequently, the solid matter was separated and purified by column chromatography to obtain 3.2 g of the objective compound (X-27N35IB-AL) represented by the following formula.

The following peaks were found by the NMR measurement performed on the obtained compound under the above measurement conditions, and the compound was confirmed to have a chemical structure of the following formula (X-27N35IB-AL).

¹H-NMR: (d6-DMSO, internal standard TMS): δ (ppm) 6.8-7.8 (12H, Ph-H), 6.1 (3H, —CH═C), 5.3-5.4 (7H, C—H, —C═CH₂), 4.8 (6H, —CH₂—)

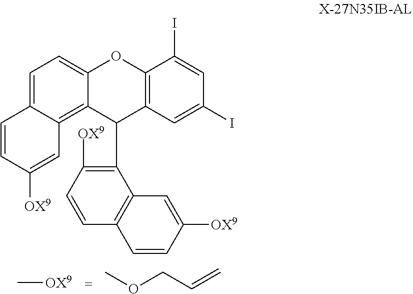

### Synthesis Working Example 1-6

### Synthesis of X-27N35IB-Ac

In the same manner as in Synthesis Working Example 1-5, except that 46.1 g (0.64 mol) of acrylic acid was used instead of the 77.6 g (0.64 mol) of allyl bromide mentioned above, 4.0 g of the objective compound (X-27N35IB-Ac) represented by the following formula was obtained.

The following peaks were found by the NMR measurement performed on the obtained compound under the above measurement conditions, and the compound was confirmed to have a chemical structure of the following formula (X-27N35IB-Ac).

¹H-NMR: (d6-DMSO, internal standard TMS): δ (ppm) 6.8-7.9 (12H, Ph-H), 6.2 (3H, ═C—H), 6.1 (3H, —CH═C), 5.7 (3H, ═C—H), 5.3 (1H, C—H)

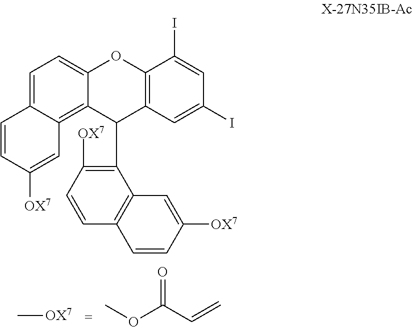

### Synthesis Working Example 1-7

### Synthesis of X-27N35IB-Ea

In a container (internal capacity: 100 ml) equipped with a stirrer, a condenser tube, and a burette, 6.6 g (10 mmol) of the compound (X-27N35IB) obtained in Synthesis Working Example 1-1, 5.5 g of glycidyl methacrylate, 0.45 g of triethylamine, and 0.08 g of p-methoxyphenol were charged in 70 ml of methyl isobutyl ketone, and the contents were warmed to 80° C. and allowed to react with stirring for 24 hours.

The resultant was cooled to 50° C., and the reaction solution was added dropwise into pure water. The precipitated solid matter was filtered, dried, and then separated and purified by column chromatography to obtain 1.8 g of the objective compound (X-27N35IB-Ea) represented by the following formula.

The obtained compound was confirmed to have a chemical structure of the following formula (X-27N35IB-Ea) by 400 MHz-¹H-NMR.

¹H-NMR: (d-DMSO, internal standard TMS): δ (ppm) 6.8-7.9 (12H, Ph-H), 6.4-6.5 (6H, C═CH₂), 5.8 (5H, —OH), 5.3 (1H, C—H), 4.7 (3H, C—H), 4.0-4.4 (12H, —CH₂—), 2.0 (9H, —CH₃)

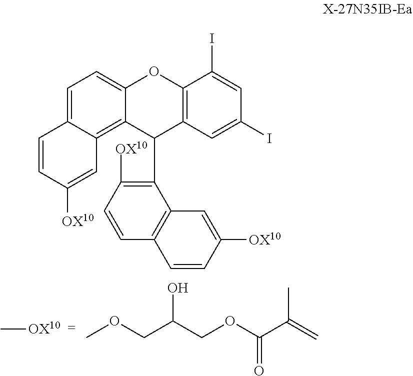

### Synthesis Working Example 1-8

### Synthesis of X-27N35IB-Ua

In a container (internal capacity: 100 mL) equipped with a stirrer, a condenser tube, and a burette, 6.6 g (10 mmol) of the compound (X-27N35IB) obtained in Synthesis Working Example 1-1, 5.5 g of 2-isocyanatoethyl methacrylate, 0.45 g of triethylamine, and 0.08 g of p-methoxyphenol were charged in 70 mL of methyl isobutyl ketone, and the contents were warmed to 80° C. and allowed to react with stirring for 24 hours. The resultant was cooled to 50° C., and the reaction solution was added dropwise into pure water. The precipitated solid matter was filtered, dried, and then separated and purified by column chromatography to obtain 1.5 g of the objective compound (X-27N35IB-Ua) represented by the following formula. The obtained compound was confirmed to have a chemical structure of the following formula (X-27N35IB-Ua) by 400 MHz-¹H-NMR.

¹H-NMR: (d-DMSO, internal standard TMS): δ (ppm) 8.8 (3H, —NH—), 6.9-8.0 (12H, Ph-H,), 6.4-6.5 (6H, ═CH₂), 5.3 (1H, C—H), 3.6-4.1 (6H, —CH₂—), 1.3-2.2 (6H, —CH₂—), 2.0 (9H, —CH₃)

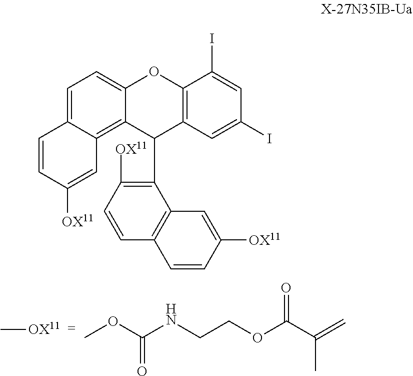

### Synthesis Working Example 1-9

### Synthesis of X-27N35IB-E

In a container (internal capacity: 200 mL) equipped with a stirrer, a condenser tube, and a burette, 6.6 g (10 mmol) of the compound (X-27N35IB) obtained in Synthesis Working Example 1-1 and 10 g (72 mmol) of potassium carbonate were charged in 60 mL of dimethylformamide, 5.0 g (40.6 mmol) of acetic acid-2-chloroethyl was added thereto, and the reaction solution was stirred at 90° C. for 12 hours and allowed to react. Next, the reaction solution was cooled in an ice bath to precipitate crystals, which were then separated by filtration. Subsequently, in a container (internal capacity: 500 mL) equipped with a stirrer, a condenser tube, and a burette, 30 g of the crystals mentioned above, 30 g of methanol, 100 g of THF, and a 24% aqueous sodium hydroxide solution were charged. The reaction solution was stirred for 4 hours under reflux and allowed to react. Then, the reaction solution was cooled in an ice bath and concentrated. The precipitated solid matter was filtered, dried, and then separated and purified by column chromatography to obtain 3.2 g of the objective compound (X-27N35IB-E) represented by the following formula. The obtained compound was confirmed to have a chemical structure of the following formula (X-27N35IB-E) by 400 MHz-¹H-NMR.

¹H-NMR: (d-DMSO, internal standard TMS): δ (ppm) 6.8-7.9 (12H, Ph-H), 5.3 (1H, C—H), 4.9 (3H, —OH), 4.4 (6H, —CH₂—), 3.7 (6H, —CH₂—)

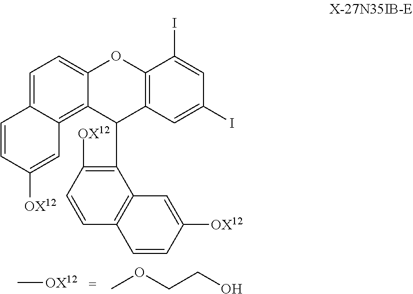

### Synthesis Working Example 1-10

### Synthesis of X-27N35IB-PX

In a container (internal capacity: 1000 mL) equipped with a stirrer, a condenser tube, and a burette, 27.6 g (42 mmol) of the compound (X-27N35IB) obtained in Synthesis Working Example 1-1, 47.2 g of iodoanisole, 87.5 g of cesium carbonate, 1.4 g of dimethylglycine hydrochloride, and 0.5 g of copper iodide were charged in 400 mL of 1,4-dioxane, and the contents were warmed to 95° C., stirred for 22 hours, and allowed to react. Next, insoluble matter was filtered off, and the filtrate was concentrated and added dropwise into pure water. The precipitated solid matter was filtered, dried, and then separated and purified by column chromatography to obtain 15 g of an intermediate compound (X-27N35IB-M) represented by the following formula.

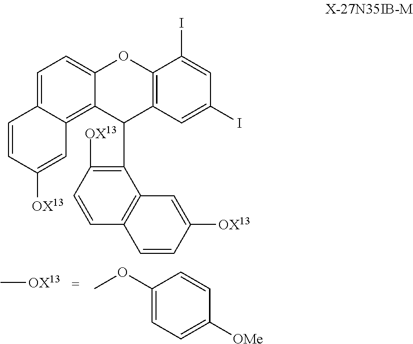

Next, in a container (internal capacity: 1000 mL) equipped with a stirrer, a condenser tube, and a burette, 10 g of the compound represented by the above formula (X-27N35IB-M) and 80 g of pyridine hydrochloride were charged, and the contents were stirred at 190° C. for 2 hours and allowed to react. Next, 160 mL of hot water was further added thereto, and the mixture was stirred to precipitate solid matter. Then, 250 mL of ethyl acetate and 100 mL of water were added thereto, and the mixture was stirred, left to stand still, and separated. The organic layer was concentrated, dried, and then separated and purified by column chromatography to obtain 6 g of the objective compound represented by the following formula (X-27N35IB-M-PX).

The obtained compound was confirmed to have a chemical structure of the following formula (X-27N35IB-PX) by 400 MHz-¹H-NMR.

¹H-NMR: (d-DMSO, internal standard TMS): δ (ppm) 9.5 (3H, O—H), 6.8-8.0 (24H, Ph-H), 5.3 (1H, C—H)

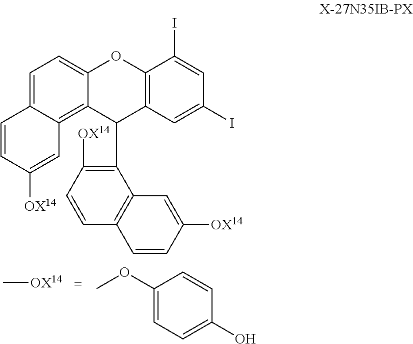

### Synthesis Working Example 1-11

### Synthesis of X-27N35IB-PE

The same reaction as in Synthesis Working Example 1-10 was performed except that the compound represented by the above formula (X-27N35IB-E) was used instead of the compound represented by the above formula (X-27N35IB), thereby obtaining 3 g of the objective compound represented by the following formula (X-27N35IB-PE).

The obtained compound was confirmed to have a chemical structure of the following formula (X-27N35IB-PE) by 400 MHz-¹H-NMR.

¹H-NMR: (d-DMSO, internal standard TMS): δ (ppm) 9.1 (3H, O—H), 6.6-7.9 (24H, Ph-H), 5.3 (1H, C—H), 4.4 (6H, —CH₂—), 3.1 (6H, —CH₂—)

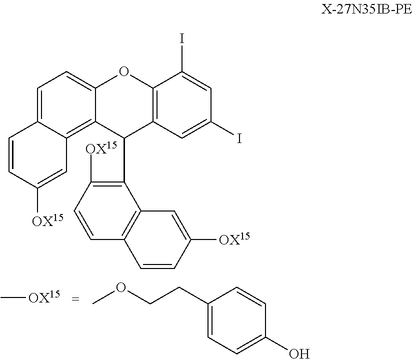

### Synthesis Working Example 1-12

### Synthesis of X-27N35IB-G

In a container (internal capacity: 100 ml) equipped with a stirrer, a condenser tube, and a burette, 5.5 g (8.4 mmol) of the compound (X-27N35IB) obtained in Synthesis Working Example 1-1 and 3.7 g (27 mmol) of potassium carbonate were charged in 100 ml of dimethylformamide, 2.5 g (27 mmol) of epichlorohydrin was further added thereto, and the resultant reaction solution was stirred at 90° C. for 6.5 hours and allowed to react. Next, solid matter was removed from the reaction solution by filtration. The reaction solution was cooled in an ice bath to precipitate crystals. The crystals were filtered, dried, and then separated and purified by column chromatography to obtain 1.8 g of the objective compound (X-27N35IB-G) represented by the following formula.

The following peaks were found by the NMR measurement performed on the obtained compound (X-27N35IB-G) under the measurement conditions mentioned above, and the compound was confirmed to have a chemical structure of the following formula (X-27N35IB-G).

¹H-NMR: (d-DMSO, internal standard TMS): δ (ppm) 6.8-7.9 (12H, Ph-H), 5.3 (C—H), 4.0 (6H, —CH₂—), 2.0-3.1 (9H, —CH(CH₂)O)

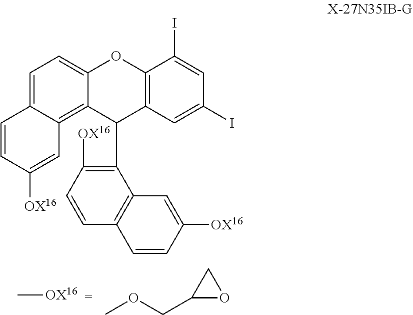

### Synthesis Working Example 1-13

### Synthesis of X-27N35IB-GE

The same reaction as in Synthesis Working Example 1-12 was performed except that the compound represented by the above formula (X-27N35IB-E) was used instead of the compound represented by the above formula (X-27N35IB), thereby obtaining 1.4 g of the objective compound represented by the following formula (X-27N35IB-GE).

The compound was confirmed to have a chemical structure of the following formula (X-27N35IB-GE) by 400 MHz-¹H-NMR.

¹H-NMR: (d-DMSO, internal standard TMS): δ (ppm) 6.8-7.9 (12H, Ph-H), 5.3 (C—H), 3.3-4.4 (18H, —CH₂—), 2.3-2.8 (9H, —CH (CH₂)O)

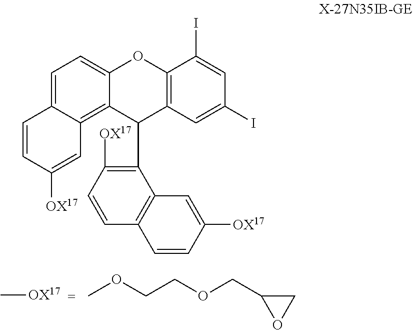

### Synthesis Working Example 1-14

### Synthesis of X-27N35IB-SX

In a container (internal capacity: 100 ml) equipped with a stirrer, a condenser tube, and a burette, 5.5 g (8.4 mmol) of the compound (X-27N35IB) obtained in Synthesis Working Example 1-1 and 3.8 g of vinyl benzyl chloride (trade name: CMS-P, manufactured by AGC SEIMI CHEMICAL CO., LTD.) were charged in 50 ml of dimethylformamide, and while warming the contents to 50° C. with stirring, 5.0 g of a 28 mass % sodium methoxide (methanol solution) was added thereto through a dropping funnel over 20 minutes. The reaction solution was stirred at 50° C. for 1 hour and allowed to react. Next, 1.0 g of a 28 mass % sodium methoxide (methanol solution) was added thereto, and the reaction solution was warmed to 60° C. and stirred for 3 hours. Furthermore, 1.0 g of an 85 mass % phosphoric acid was added thereto, and after stirring for 10 minutes, the reaction solution was cooled to 40° C. and added dropwise into pure water. The precipitated solid matter was filtered, dried, and then separated and purified by column chromatography to obtain 1.9 g of the objective compound (X-27N35IB-SX) represented by the following formula.

The obtained compound was confirmed to have a chemical structure of the following formula (X-27N35IB-SX) by 400 MHz-¹H-NMR.

¹H-NMR: (d-DMSO, internal standard TMS): δ (ppm) 6.9-7.9 (24H, Ph-H), 6.7 (3H, —CH═C), 5.8 (3H, —C═CH), 5.2-5.3 (10H, —CH₂—, —C═CH, C—H)

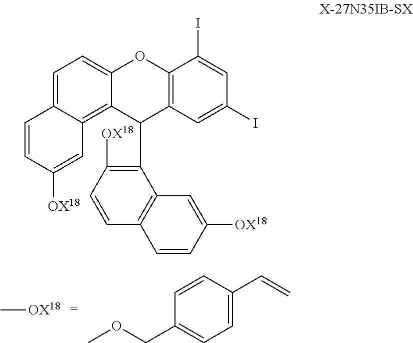

### Synthesis Working Example 1-15

### Synthesis of X-27N35IB-SE

The same reaction as in Synthesis Working Example 1-14 was performed except that the compound represented by the above formula (X-27N35IB-E) was used instead of the compound represented by the above formula (X-27N35IB), thereby obtaining 1.9 g of the objective compound (X-27N35IB-SE) represented by the following formula.

The obtained compound was confirmed to have a chemical structure of the following formula (X-27N35IB-SE) by 400 MHz-¹H-NMR.

¹H-NMR: (d-DMSO, internal standard TMS): δ (ppm) 6.7-7.9 (24H, Ph-H), 6.7 (3H, —CH═C), 5.8 (3H, —C═CH), 5.3 (4H, C—H, —C═CH), 4.8 (6H, —CH₂—), 4.4 (6H, —CH₂—), 3.8 (6H, —CH₂—)

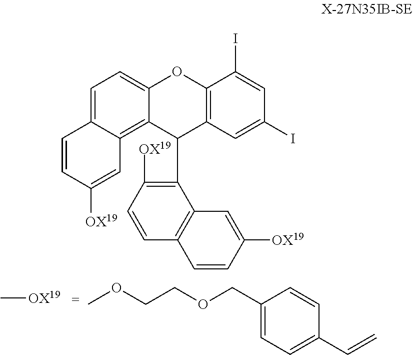

### Synthesis Working Example 1-16

### Synthesis of X-27N35IB-Pr

In a container (internal capacity: 300 mL) equipped with a stirrer, a condenser tube, and a burette, 5.5 g (8.4 mmol) of the compound (X-27N35IB) obtained in Synthesis Working Example 1-1 and 4.8 g (40 mmol) of propargyl bromide were charged in 100 mL of dimethylformamide, and the contents were allowed to react by being stirred at room temperature for 3 hours to obtain a reaction solution. Next, the reaction solution was concentrated, and the reaction product was precipitated by the addition of 300 g of pure water to the concentrate, cooled to room temperature, and then filtered to separate solid matter.

The obtained solid matter was filtered and then dried. Subsequently, the solid matter was separated and purified by column chromatography to obtain 3.0 g of the objective compound (X-27N35IB-Pr) represented by the following formula.

The following peaks were found by the NMR measurement performed on the obtained compound (X-27N35IB-Pr) under the measurement conditions mentioned above, and the compound was confirmed to have a chemical structure of the following formula (X-27N35IB-Pr).

¹H-NMR: (d-DMSO, internal standard TMS): δ (ppm): 6.9-7.9 (12H, Ph-H), 5.3 (1H, C—H), 4.8 (6H, —CH₂—), 3.4 (3H, ≡CH)

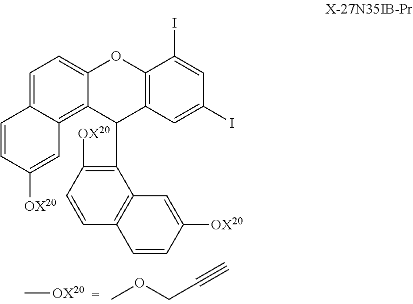

### Synthesis Working Example 2

### Synthesis of X-27NSA

The same reaction as in Synthesis Working Example 1-1 was performed except that 8.67 g (71 mmol) of salicylaldehyde was used instead of 25.4 g (71 mmol) of 3,5-diiodosalicylaldehyde, thereby obtaining 2.2 g of the objective compound (X-27NSA) represented by the following formula.

The obtained compound was confirmed to have a chemical structure of the following formula (X-27NSA) by 400 MHz-¹H-NMR.

¹H-NMR: (d-DMSO, internal standard TMS): δ (ppm) 9.2-9.7 (3H, —O—H), 6.8-7.8 (14H, Ph-H), 5.3 (1H, C—H)

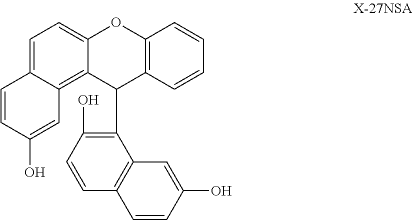 [SMILES: Oc1ccc2ccc(O)c(C3c4ccccc4Oc4ccc5ccc(O)cc5c43)c2c1]

### Synthesis Working Example 3

### Synthesis of X-27N4PSA

The same reaction as in Synthesis Working Example 1-1 was performed except that 14.1 g (71 mmol) of 4-phenylsalicylaldehyde was used instead of 25.4 g (71 mmol) of 3,5-diiodosalicylaldehyde, thereby obtaining 1.7 g of the objective compound represented by the following formula (X-27N4PSA).

The obtained compound was confirmed to have a chemical structure of the following formula (X-27N4PSA) by 400 MHz-¹H-NMR.

¹H-NMR: (d-DMSO, internal standard TMS): δ (ppm) 9.2-9.7 (3H, —O—H), 6.8-7.8 (18H, Ph-H), 5.3 (1H, C—H)

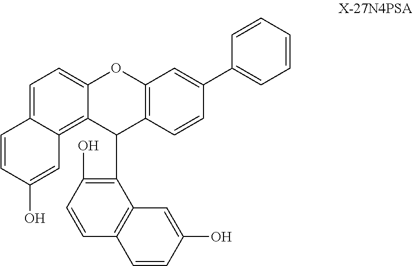 [SMILES: Oc1ccc2ccc(O)c(C3c4ccc(-c5ccccc5)cc4Oc4ccc5ccc(O)cc5c43)c2c1]

### Synthesis Working Example 4

### Synthesis of X-26NSA

The same reaction as in Synthesis Working Example 2 was performed except that 2,6-dihydroxynaphthalene was used instead of 2,7-dihydroxynaphthalene, thereby obtaining 1.4 g of the objective compound (X-26NSA) represented by the following formula (X-26NSA).

The obtained compound was confirmed to have a chemical structure of the following formula (X-26NSA) by 400 MHz-¹H-NMR.

1H-NMR: (d-DMSO, internal standard TMS): δ (ppm) 9.2-9.7 (3H, —O—H), 6.7-7.8 (14H, Ph-H), 5.3 (1H, C—H)

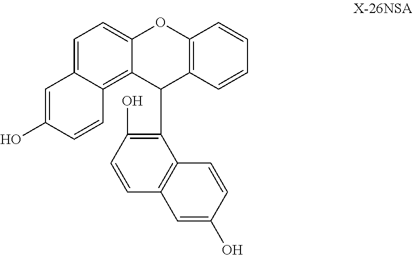 [SMILES: Oc1ccc2c(C3c4ccccc4Oc4ccc5cc(O)ccc5c43)c(O)ccc2c1]

### Synthesis Comparative Example 1

### Synthesis of AC-1

4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to prepare a reaction solution. This reaction solution was polymerized for 22 hours with the reaction temperature kept at 63° C. in a nitrogen atmosphere. Then, the reaction solution was added dropwise into 400 mL of n-hexane. The obtained product resin was solidified and purified, and the resulting white powder was filtered and then dried overnight at 40° C. under reduced pressure to obtain AC-1 represented by the following formula.

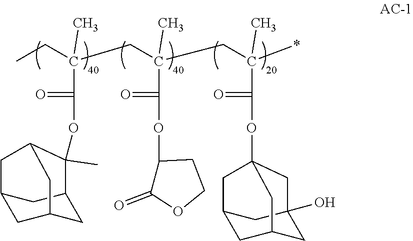

In the formula AC-1, “40”, “40”, and “20” represent the ratio of each constituent unit and do not represent a block copolymer.

### [Evaluation Method]

### (1) Safe Solvent Solubility Test of Compound

The solubility of a compound in PGME, PGMEA, and CHN was evaluated according to the following criteria, using the amount of dissolution of the compound in each solvent. The amount of dissolution was measured at 23° C. by precisely weighing the compound into a test tube, adding the target solvent so as to attain a predetermined concentration, applying ultrasonic waves for 30 minutes in an ultrasonic cleaner, and then visually observing the subsequent state of the fluid.

A: 5.0% by mass≤Amount of dissolution

B: 2.0% by mass≤Amount of dissolution <5.0% by mass

C: Amount of dissolution <2.0% by mass

### (2) Storage Stability and Thin Film Formation of Resist Composition

The storage stability of a resist composition containing the compound was evaluated by leaving the resist composition after fabrication to stand still at 23° C. for 3 days, and visually observing the presence or absence of precipitates. A clean silicon wafer was spin coated with the resist composition, and then prebaked (PB) before exposure on a hot plate at 110° C. to form a resist film with a thickness of 50 nm. The fabricated resist composition was evaluated as “A” when it was a homogeneous solution and the thin film formation went well, “B” when it was a homogeneous solution but the thin film had defects, and “C” when precipitates were observed.

### (3) Pattern Evaluation of Resist Pattern (Pattern Formation)

The resist film obtained in the above (2) was irradiated with electron beams of 1:1 line and space setting with a 50 nm interval using an electron beam lithography system (ELS-7500 manufactured by ELIONIX INC.).

After the irradiation, the resist film was heated at 110° C. for 90 seconds, and immersed in a 2.38 mass % TMAH alkaline developing solution for 60 seconds for development. Subsequently, the resist film was washed with ultrapure water for 30 seconds, and dried to form a resist pattern.

The shape of the obtained resist pattern of 50 nm L/S (1:1) was observed using an electron microscope (5-4800) manufactured by Hitachi Ltd. The “resist pattern shape” after the development was evaluated as “A” when having better rectangularity without pattern collapse compared to Comparative Example 1, and evaluated as “C” when it was equivalent to or inferior to Comparative Example 1.

Furthermore, the smallest electron beam energy quantity capable of lithographing a good pattern shape was set as “sensitivity”, and those in which the sensitivity is superior to Comparative Example 1 by 10% or more were evaluated to be “S”, those in which the sensitivity is superior by less than 10% were evaluated to be “A”, and those in which the sensitivity is equivalent to or inferior to Comparative Example 1 were evaluated to be “C”.

### (4) Etching Resistance

Etching apparatus: RIE-10NR manufactured by Samco International, Inc.

Output: 50 W

Pressure: 20 Pa

Time: 2 min

Etching gas

Ar gas flow rate:CF₄ gas flow rate:O₂ gas flow rate=50:5:5 (sccm)

For each of the films obtained in Examples and Comparative Example, an etching test was carried out under the conditions mentioned above, and the etching rate upon that time was measured. Then, the etching resistance was evaluated according to the following evaluation criteria on the basis of the etching rate of an underlayer film fabricated by using a novolac (“PSM4357” manufactured by Gun Ei Chemical Industry Co., Ltd.).

### Evaluation Criteria

A: The etching rate was smaller as compared with the underlayer film of novolac.

C: The etching rate was larger as compared with the underlayer film of novolac.

For the compounds obtained in the above Synthesis Working Examples 1-1 to 1-16, 2, 3, and 4, and Synthesis Comparative Example 1, the results of evaluating their solubilities in safe solvents by the method described above are shown in Table 1.

TABLE 1

| Compound | PGME | PGMEA | CHN |
|---|---|---|---|
| Synthesis Working Example 1-1 | A | A | A |
| Synthesis Working Example 1-2 | A | A | A |
| Synthesis Working Example 1-3 | A | A | A |
| Synthesis Working Example 1-4 | A | A | A |
| Synthesis Working Example 1-5 | A | A | A |
| Synthesis Working Example 1-6 | A | A | A |
| Synthesis Working Example 1-7 | A | A | A |
| Synthesis Working Example 1-8 | A | A | A |
| Synthesis Working Example 1-9 | A | A | A |
| Synthesis Working Example 1-10 | A | A | A |
| Synthesis Working Example 1-11 | A | A | A |
| Synthesis Working Example 1-12 | A | A | A |
| Synthesis Working Example 1-13 | A | A | A |
| Synthesis Working Example 1-14 | A | A | A |
| Synthesis Working Example 1-15 | A | A | A |
| Synthesis Working Example 1-16 | A | A | A |
| Synthesis Working Example 2 | A | A | A |
| Synthesis Working Example 3 | A | A | A |
| Synthesis Working Example 4 | A | A | A |
| Synthesis Comparative Example 1 | A | A | A |

### Examples 1 to 23 and Comparative Example 1

Compositions for lithography were each prepared according to the composition shown in Table 2 below.

Next, a silicon substrate was spin coated with each of these compositions for lithography, and then baked at 110° C. for 90 seconds to fabricate each resist film with a film thickness of 50 nm. The following acid generating agent, acid diffusion controlling agent, and organic solvent were used.

Acid generating agent: triphenylsulfonium nonafluoromethanesulfonate (TPS-109) manufactured by Midori Kagaku Co., Ltd.

Acid diffusion controlling agent: tri-n-octylamine (TOA) manufactured by Kanto Chemical Co., Inc.

Crosslinking agent: NIKALAC MW-100LM manufactured by Sanwa Chemical Co., Ltd.

Organic solvent: propylene glycol monomethyl ether (PGME) manufactured by Kanto Chemical Co., Inc.

TABLE 2

| | Resist | | | Acid | | |
|---|---|---|---|---|---|---|
| | base | | Acid | diffusion | | |
| | material | | generating | controlling | Crosslinking | Organic |
| | (A) | Polyphenol | agent | agent | agent | solvent |
| | (parts by | compound (B) | (parts by | (parts by | (parts by | (parts by |
| | mass) | (parts by mass) | mass) | mass) | mass) | mass) |
| Example 1 | None | X-27N35IB | TPS-109 | TOA | MW-100LM | PGME |
| | | 5 | 1 | 0.1 | 1 | 120 |
| Example 2 | None | X-27N35IB-MeBOC | TPS-109 | TOA | None | PGME |
| | | 5 | 1 | 0.1 | | 100 |
| Example 3 | None | R-X-27N35IB | TPS-109 | TOA | None | PGME |
| | | 5 | 1 | 0.1 | | 100 |
| Example 4 | AC-1 | X-27N35IB | TPS-109 | TOA | None | PGME |
| | 4.5 | 0.5 | 1 | 0.1 | | 100 |
| Example 5 | AC-1 | X-27N35IB-MeBOC | TPS-109 | TOA | None | PGME |
| | 4.5 | 0.5 | 1 | 0.1 | | 100 |
| Example 6 | AC-1 | R-X-27N35IB | TPS-109 | TOA | None | PGME |
| | 4.5 | 0.5 | 1 | 0.1 | | 100 |
| Example 7 | None | X-27N35IB-BOC | TPS-109 | TOA | None | PGME |
| | | 5 | 1 | 0.1 | | 100 |
| Example 8 | None | X-27N35IB-AL | TPS-109 | TOA | None | PGME |
| | | 5 | 1 | 0.1 | | 100 |
| Example 9 | None | X-27N35IB-Ac | TPS-109 | TOA | None | PGME |
| | | 5 | 1 | 0.1 | | 100 |
| Example 10 | None | X-27N35IB-Ea | TPS-109 | TOA | None | PGME |
| | | 5 | 1 | 0.1 | | 100 |
| Example 11 | None | X-27N35IB-Ua | TPS-109 | TOA | None | PGME |
| | | 5 | 1 | 0.1 | | 100 |
| Example 12 | None | X-27N35IB-E | TPS-109 | TOA | None | PGME |
| | | 5 | 1 | 0.1 | | 100 |
| Example 13 | None | X-27N35IB-M | TPS-109 | TOA | None | PGME |
| | | | 1 | 0.1 | | 100 |
| Example 14 | None | X-27N35IB-PX | TPS-109 | TOA | None | PGME |
| | | 5 | 1 | 0.1 | | 100 |
| Example 15 | None | X-27N35IB-PE | TPS-109 | TOA | None | PGME |
| | | 5 | 1 | 0.1 | | 100 |
| Example 16 | None | X-27N35IB-G | TPS-109 | TOA | None | PGME |
| | | 5 | 1 | 0.1 | | 100 |
| Example 17 | None | X-27N35IB-GE | TPS-109 | TOA | None | PGME |
| | | 5 | 1 | 0.1 | | 100 |
| Example 18 | None | X-27N35IB-SX | TPS-109 | TOA | None | PGME |
| | | 5 | 1 | 0.1 | | 100 |
| Example 19 | None | X-27N35IB-SE | TPS-109 | TOA | None | PGME |
| | | 5 | 1 | 0.1 | | 100 |
| Example 20 | None | X-27N35IB-Pr | TPS-109 | TOA | None | PGME |
| | | 5 | 1 | 0.1 | | 100 |
| Example 21 | None | X-27NSA | TPS-109 | TOA | MW-100LM | PGME |
| | | 5 | 1 | 0.1 | 1 | 120 |
| Example 22 | None | X-27N4PSA | TPS-109 | TOA | MW-100LM | PGME |
| | | 5 | 1 | 0.1 | 1 | 120 |
| Example 23 | None | X-26NSA | TPS-109 | TOA | MW-100LM | PGME |
| | | 5 | 1 | 0.1 | 1 | 120 |
| Comparative | AC-1 | None | TPS-109 | TOA | None | PGME |
| Example 1 | 5 | | 1 | 0.1 | | 100 |

Next, each composition was evaluated by the methods mentioned above. The evaluation results are shown in Table 3.

TABLE 3

| | Storage stability | | | |
|---|---|---|---|---|
| | and thin film | Resist | | Etching |
| | formation | pattern | Sensitivity | resistance |
| Example 1 | A | A | S | A |
| Example 2 | A | A | S | A |
| Example 3 | A | A | A | A |
| Example 4 | A | A | S | A |
| Example 5 | A | A | S | A |
| Example 6 | A | A | A | A |
| Example 7 | A | A | S | A |
| Example 8 | A | A | S | A |
| Example 9 | A | A | S | A |
| Example 10 | A | A | S | A |
| Example 11 | A | A | S | A |
| Example 12 | A | A | S | A |
| Example 13 | A | A | S | A |
| Example 14 | A | A | S | A |
| Example 15 | A | A | S | A |
| Example 16 | A | A | S | A |
| Example 17 | A | A | S | A |
| Example 18 | A | A | S | A |
| Example 19 | A | A | S | A |
| Example 20 | A | A | S | A |
| Example 21 | A | A | A | A |
| Example 22 | A | A | A | A |
| Example 23 | A | A | A | A |
| Comparative | A | C | C | C |
| Example 1 | | | | |

### Examples 24 to 27

### <Measurement of EUV Absorption Rate>

The compound (X-27N35IB) of Synthesis Working Example 1 was dissolved in PGME and a silicon substrate was spin coated with this. Then, it was baked at 110° C. for 90 seconds to fabricate a film having a film thickness of 50 nm (Example 24). In the same manner, the films of Example 25, Example 26, and Example 27 were fabricated using the compound of Synthesis Working Example 2 (X-27NSA), the compound of Synthesis Working Example 3 (X-27N4PSA), and the compound of Synthesis Working Example 4 (X-26NSA), respectively.

These films were measured for film density under the conditions shown below. A film density of 1.7 or more was defined as A, 1.4 or more and less than 1.7 as B, and less than 1.4 as C. The measurement results are shown in Table 4.

### (Film Density Measurement Conditions)

Apparatus name: X-ray diffractometer manufactured by PANalytical B.V.

Voltage and current: 45 kV and 40 mA

X-ray wavelength: CuKα1 radiation

Incidence spectrometer: X-ray focusing mirror+Ge220 double-crystal

Analytical software: LEPTOS 6.02 manufactured by Bruker AXS

From the film density obtained as described above and the mass absorption coefficient of the constituent elements, the EUV absorption rate per 40 nm was calculated. The calculated coefficients are shown in Table 4. Note that, for the calculation of the EUV absorption rate, the following websites of Lawrence Berkeley National Laboratory, USA, were used. http://henke.lbl.gov/optical_constants/ http://henke.lbl.gov/optical_constants/filter2.html An EUV absorption rate of 30% or more when transmitting 40 nm was defined as A, 20% or more and less than 30% as B, and less than 20% as C.

### <Measurement of Refractive Index>

The compound (X-27N35IB) of Synthesis Working Example 1 was dissolved in PGME and a clean silicon wafer was spin coated with this. Then, it was baked in an oven at 110° C. to form a film having a thickness of 1 μm. The refractive index (λ=550 nm) of the film at 25° C. was measured using a variable angle spectroscopic ellipsometer VASE manufactured by J. A. Woollam Co., Inc. The prepared film was evaluated as “A” when the refractive index was 1.70 or more, evaluated as “B” when the refractive index was 1.65 or more and less than 1.70, and evaluated as “C” when the refractive index was less than 1.65. The measurement results are shown in Table 4.

### Comparative Examples 2 to 5

Except that polyhydroxystyrene (Mw: 8000, manufactured by Sigma-Aldrich), which is a general resist material, and compounds represented by the following formulas (PP-1) and (PP-2), synthesized by the methods described in WO 2016/158168, were used instead of the compound (X-27N35IB) in Example 24, the film density, EUV absorption rate, and refractive index were measured in the same manner as in Example 24. The results are shown in Table 4.

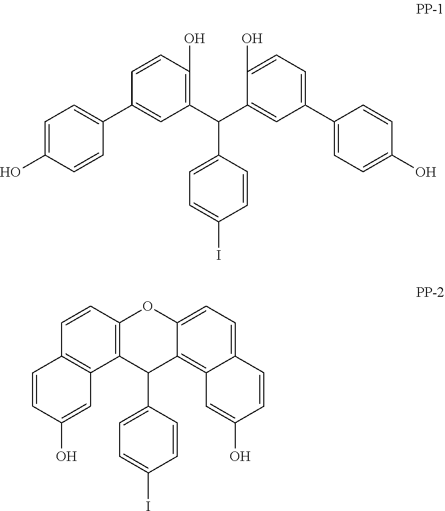 [SMILES: Oc1ccc(-c2ccc(O)c(C(c3ccc(I)cc3)c3cc(-c4ccc(O)cc4)ccc3O)c2)cc1 ; Oc1ccc2ccc3c(c2c1)C(c1ccc(I)cc1)c1c(ccc2ccc(O)cc12)O3]

In the same manner as in Synthesis Working Example 4 except that 4-hydroxybenzaldehyde was used instead of salicylaldehyde, a compound represented by the following formula (PP-3) was synthesized. Using this PP-3, the film density, EUV absorption rate, and refractive index were measured in the same manner as in Example 24. The results are shown in Table 4.

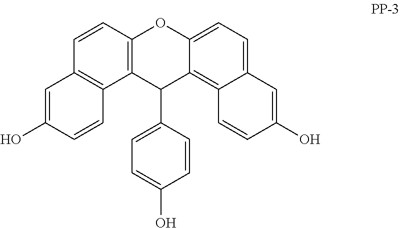 [SMILES: Oc1ccc(C2c3c(ccc4cc(O)ccc34)Oc3ccc4cc(O)ccc4c32)cc1]

TABLE 4

| | | | EUV | |
|---|---|---|---|---|
| | | Film | absorption | Refractive |
| | Compound | density | rate | index |
| Example 24 | X-27N35IB | A | A | A |
| Example 25 | X-27NSA | A | B | A |
| Example 26 | X-27N4PSA | A | B | A |
| Example 27 | X-26NSA | A | B | A |
| Comparative | Polyhydroxystyrene | C | C | C |
| Example 2 | (Mw: 8000) | | | |
| Comparative | PP-1 | B | B | B |
| Example 3 | | | | |
| Comparative | PP-2 | B | B | B |
| Example 4 | | | | |
| Comparative | PP-3 | B | B | B |
| Example 5 | | | | |

### Examples 28 to 50 and Comparative Example 6

### <Synthesis of MAR1>

0.5 g of the compound AR1, 3.0 g of 2-methyl-2-adamantyl methacrylate, 2.0 g of γ-butyrolactone methacrylate, and 1.5 g of hydroxyadamantyl methacrylate were dissolved in 45 mL of tetrahydrofuran, and 0.20 g of azobisbutyronitrile was added thereto. After refluxing for 12 hours, the reaction solution was added dropwise into 2 L of n-heptane. The precipitated polymer was filtered off and dried under reduced pressure, thereby obtaining MAR1, a white powdery polymer represented by the following formula (MAR1). This polymer had a weight average molecular weight (Mw) of 12,000 and a dispersity (Mw/Mn) of 1.90. Also, as a result of measuring the ¹³C-NMR, the compositional ratio (molar ratio) in the following formula (MAR1) was a:b:c:d=40:30:15:15. Note that, although the following formula (MAR1) is described in a simplified form to show the ratio of each constituent unit, the arrangement order of each constituent unit is random and the polymer is not a block copolymer in which each constituent unit forms a block independent of each other. The molar ratio was determined based on the integral ratio of the root carbon of the benzene ring for the polystyrene based monomer (compound AR1), and the respective carbonyl carbons of the ester bonds for the methacrylate based monomers (2-methyl-2-adamantyl methacrylate, γ-butyrolactone methacrylate, and hydroxyadamantyl methacrylate).

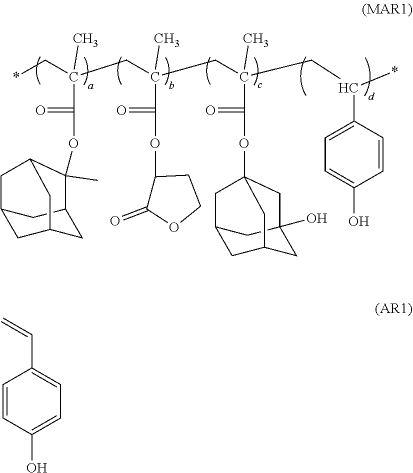

### (Fabrication of Resist Solution for EUV Sensitivity Evaluation)

A solution was prepared by compounding 5 parts by mass of the fabricated polymer MAR1, 1 part by mass of triphenylsulfonium nonafluoromethanesulfonate, 0.2 parts by mass of tributylamine, 80 parts by mass of PGMEA, and 12 parts by mass of PGME.

### (Fabrication of Underlayer Film Forming Solution)

Underlayer film forming solutions were prepared by the same method as in Example 1, except that the materials described in Table 5 were used, and the underlayer films of Examples 28 to 50 and Comparative Example 6 were fabricated.

TABLE 5

| | | | Acid | | Organic |
|---|---|---|---|---|---|
| | Resist base | Polyphenol | generating | Crosslinking | solvent |
| | material (A) | compound (B) | agent | agent | PGMEA/PGME |
| | (parts by mass) | (parts by mass) | (parts by mass) | (parts by mass) | (parts by mass) |
| Example 28 | None | X-27N35IB | TPS-109 | MW-100LM | 50/10 |
| | | 5 | 1 | 1 | |
| Example 29 | None | X-27N35IB-MeBOC | TPS-109 | MW-100LM | 50/10 |
| | | 5 | 1 | 1 | |
| Example 30 | None | R-X-27N35IB | TPS-109 | MW-100LM | 50/10 |
| | | 5 | 1 | 1 | |
| Example 31 | AC-1 | X-27N35IB | TPS-109 | MW-100LM | 50/10 |
| | 4 | 1 | 1 | 1 | |
| Example 32 | AC-1 | X-27N35IB-MeBOC | TPS-109 | MW-100LM | 50/10 |
| | 4 | 1 | 1 | 1 | |
| Example 33 | AC-1 | R-X-27N35IB | TPS-109 | MW-100LM | 50/10 |
| | 4 | 1 | 1 | 1 | |
| Example 34 | None | X-27N35IB-BOC | TPS-109 | MW-100LM | 50/10 |
| | | 5 | 1 | 1 | |
| Example 35 | None | X-27N35IB-AL | TPS-109 | None | 50/10 |
| | | 5 | 1 | | |
| Example 36 | None | X-27N35IB-AC | TPS-109 | None | 50/10 |
| | | 5 | 1 | | |
| Example 37 | None | X-27N35IB-Ea | TPS-109 | None | 50/10 |
| | | 5 | 1 | | |
| Example 38 | None | X-27N35IB-Ua | TPS-109 | None | 50/10 |
| | | 5 | 1 | | |
| Example 39 | None | X-27N35IB-E | TPS-109 | None | 50/10 |
| | | 5 | 1 | | |
| Example 40 | None | X-27N35IB-M | TPS-109 | None | 50/10 |
| | | 5 | 1 | | |
| Example 41 | None | X-27N35IB-PX | TPS-109 | None | 50/10 |
| | | 5 | 1 | | |
| Example 42 | None | X-27N35IB-PE | TPS-109 | None | 50/10 |
| | | 5 | 1 | | |
| Example 43 | None | X-27N35IB-G | TPS-109 | None | 50/10 |
| | | 5 | 1 | | |
| Example 44 | None | X-27N35IB-GE | TPS-109 | None | 50/10 |
| | | 5 | 1 | | |
| Example 45 | None | X-27N35IB-SX | TPS-109 | None | 50/10 |
| | | 5 | 1 | | |
| Example 46 | None | X-27N35IB-SE | TPS-109 | None | 50/10 |
| | | 5 | 1 | | |
| Example 47 | None | X-27N35IB-Pr | TPS-109 | None | 50/10 |
| | | 5 | 1 | | |
| Example 48 | None | X-27NSA | TPS-109 | MW-100LM | 50/10 |
| | | 5 | 1 | 1 | |
| Example 49 | None | X-27N4PSA | TPS-109 | MW-100LM | 50/10 |
| | | 5 | 1 | 1 | |
| Example 50 | None | X-26NSA | TPS-109 | MW-100LM | 50/10 |
| | | 5 | 1 | 1 | |
| Comparative | AC-1 | None | TPS-109 | MW-100LM | 50/10 |
| Example 6 | 5 | | 1 | 1 | |

### [Evaluation]

The underlayer films obtained from the compounds or polymers obtained in the Examples 28 to 50 and Comparative Example 6 mentioned above were evaluated as follows. The results are shown in Table 6.

### (Evaluation of EUV Sensitivity)

A silicon wafer was coated with each of the underlayer film forming solutions fabricated in Examples 28 to 50 and further subjected to a heat treatment on a hot plate under conditions of 240° C. for 1 minute, thereby forming a wafer with an underlayer film, the wafer having an underlayer film with a thickness of 100 nm formed thereon.

Furthermore, the fabricated wafer with an underlayer film was coated with the resist solution for EUV sensitivity evaluation prepared as mentioned above, and baked at 110° C. for 60 seconds to form a photoresist layer with a film thickness of 70 nm.

Next, the wafer was subjected to maskless shot exposure with an extreme ultraviolet (EUV) exposure apparatus “EUVES-7000” (product name, manufactured by Litho Tech Japan Corporation) with the amount of exposure increased from 1 mJ/cm² to 80 mJ/cm² in increments of 1 mJ/cm², followed by baking at 110° C. for 90 seconds (PEB). Then, development was performed with a 2.38 mass % tetramethylammonium hydroxide (TMAH) aqueous solution for 60 seconds, thereby obtaining a wafer onto which shot exposure of 80 shots had been carried out. For each shot exposure area obtained, the film thickness was measured using an optical interference film thickness meter “OPTM” (product name, manufactured by Otsuka Electronics Co., Ltd.), and the profile data of the film thickness relative to the amount of exposure was acquired. The amount of exposure at which the slope of the film thickness variation amount relative to the amount of exposure is the largest was calculated as the sensitivity value (mJ/cm²) and used as an index for the EUV sensitivity of the resist.

TABLE 6

| | Resist base | Polyphenol | Evaluation |
|---|---|---|---|
| | material (A) | compound (B) | of EUV |
| | (parts by mass) | (parts by mass) | sensitivity |
| Example 28 | None | X-27N35IB | 25 |
| | | 5 | |
| Example 29 | None | X-27N35IB-MeBOC | 29 |
| | | 5 | |
| Example 30 | None | R-X-27N35IB | 26 |
| | | 5 | |
| Example 31 | AC-1 | X-27N35IB | 34 |
| | 4 | 1 | |
| Example 32 | AC-1 | X-27N35IB-MeBOC | 36 |
| | 4 | 1 | |
| Example 33 | AC-1 | R-X-27N35IB | 34 |
| | 4 | 1 | |
| Example 34 | None | X-27N35IB-BOC | 28 |
| | | 5 | |
| Example 35 | None | X-27N35IB-AL | 26 |
| | | 5 | |
| Example 36 | None | X-27N35IB-AC | 27 |
| | | 5 | |
| Example 37 | None | X-27N35IB-Ea | 28 |
| | | 5 | |
| Example 38 | None | X-27N35IB-Ua | 28 |
| | | 5 | |
| Example 39 | None | X-27N35IB-E | 27 |
| | | 5 | |
| Example 40 | None | X-27N35IB-M | 27 |
| | | 5 | |
| Example 41 | None | X-27N35IB-PX | 29 |
| | | 5 | |
| Example 42 | None | X-27N35IB-PE | 28 |
| | | 5 | |
| Example 43 | None | X-27N35IB-G | 26 |
| | | 5 | |
| Example 44 | None | X-27N35IB-GE | 27 |
| | | 5 | |
| Example 45 | None | X-27N35IB-SX | 29 |
| | | 5 | |
| Example 46 | None | X-27N35IB-SE | 30 |
| | | 5 | |
| Example 47 | None | X-27N35IB-Pr | 26 |
| | | 5 | |
| Example 48 | None | X-27NSA | 38 |
| | | 5 | |
| Example 49 | None | X-27N4PSA | 38 |
| | | 5 | |
| Example 50 | None | X-26NSA | 37 |
| | | 5 | |
| Comparative | AC-1 | None | 42 |
| Example 6 | 5 | | |

From the compounds of the present embodiment, films were obtained that have a high film density, a high EUV absorption and EUV sensitivity, and a high refractive index.

As mentioned above, the compositions comprising the compounds of the present embodiment are resist compositions that is capable of maintaining good storage stability and thin film formability, having high sensitivity and high etching resistance, and imparting a good shape to a resist pattern. In addition, the compositions comprising the compounds of the present embodiment can be used to produce underlayer films and the like that have effects of enhancing the EUV sensitivity of resists. Moreover, the compounds of the present embodiment are capable of forming films with a high density.

Therefore, when these compounds and the like are used in compositions for film formation purposes for photography or film formation purposes for resist, it is possible to form films having high resolution and high sensitivity, and they can be widely and effectively utilized in a variety of applications where these performances are required.

The present application is based on Japanese Patent Application No. 2018-248508 filed on Dec. 28, 2018, the contents of which are incorporated herein by reference.

### INDUSTRIAL APPLICABILITY

The compound and composition of the present invention have industrial applicability as compositions for film formation purposes for photography and film formation purposes for resist, and as a variety of optical component materials.

1. A compound comprising a condensed skeleton of an aromatic compound represented by formula (1-1) and an aromatic aldehyde represented by formula (2-1): 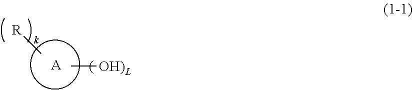
wherein
A represents an aromatic ring;
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
k is an integer of 0 or more; and
L is an integer of 1 or more, and 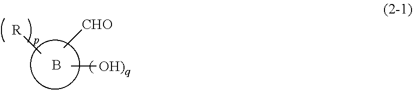
wherein
B represents an aromatic ring;
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
p is an integer of 0 or more; and
q is an integer of 1 or more,
provided that at least one hydroxy group is bonded to a carbon atom adjacent to a carbon atom to which a formyl group is bonded.

2. The compound according to claim 1, wherein the condensed skeleton has asymmetry.

3. The compound according to claim 1, wherein the condensed skeleton is represented by formula (3-1): 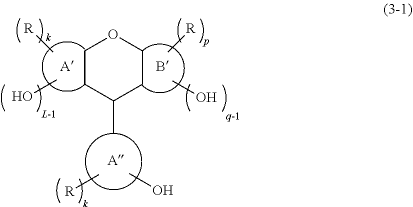
wherein
A′ and A″ are the same as A in the above formula (1-1);
B′ is the same as B in the above formula (2-1);
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
L is an integer of 1 or more;
p is an integer of 0 or more;
q is an integer of 1 or more; and
k is an integer of 0 or more.

4. The compound according to claim 1, wherein:
the aromatic compound represented by the formula (1-1) is a compound of the following formula (1-2); and
the aromatic aldehyde represented by the formula (2-1) is a compound of the following formula (2-2), 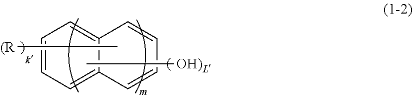
wherein
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
m is an integer of 0 to 3;
k′ is an integer of 0 to 5 when m=0, an integer of 0 to 7 when m=1, an integer of 0 to 9 when m=2, or an integer of 0 to 11 when m=3; and
L′ is an integer of 1 to 5 when m=0, an integer of 1 to 7 when m=1, an integer of 1 to 9 when m=2, or an integer of 1 to 11 when m=3, and 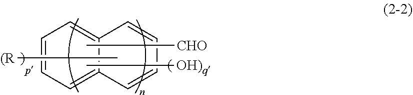
wherein
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
n is an integer of 0 to 3;
p′ is an integer of 0 to 4 when n=0, an integer of 0 to 6 when n=1, an integer of 0 to 8 when n=2, or an integer of 0 to 10 when n=3; and
q′ is an integer of 1 to 5 when n=0, an integer of 1 to 7 when n=1, an integer of 1 to 9 when n=2, or an integer of 1 to 11 when n=3.

5. The compound according to claim 1, wherein the condensed skeleton is represented by the following formula (3-2): 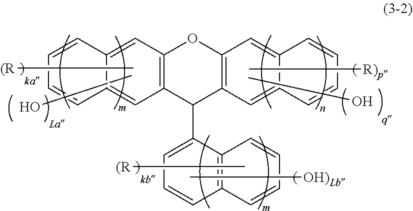
wherein
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
m is an integer of 0 to 3;
n is an integer of 0 to 3;
ka″ is an integer of 0 to 4 when m=0, an integer of 0 to 6 when m=1, an integer of 0 to 8 when m=2, or an integer of 0 to 10 when m=3;
La″ is an integer of 0 to 4 when m=0, an integer of 0 to 6 when m=1, an integer of 0 to 8 when m=2, or an integer of 0 to 10 when m=3;
kb″ is an integer of 0 to 5 when m=0, an integer of 0 to 7 when m=1, an integer of 0 to 9 when m=2, or an integer of 0 to 11 when m=3;
Lb″ is an integer of 0 to 5 when m=0, an integer of 0 to 7 when m=1, an integer of 0 to 9 when m=2, or an integer of 0 to 11 when m=3;
p″ is an integer of 0 to 4 when n=0, an integer of 0 to 6 when n=1, an integer of 0 to 8 when n=2, or an integer of 0 to 10 when n=3; and
q″ is an integer of 0 to 4 when n=0, an integer of 0 to 6 when n=1, an integer of 0 to 8 when n=2, or an integer of 0 to 10 when n=3.

6. The compound according to claim 1, wherein the condensed skeleton is represented by the following formula (3-3): 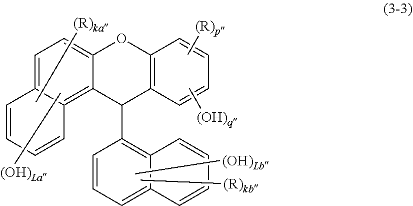
wherein
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
ka″ is an integer of 0 to 6;
La″ is an integer of 0 to 6;
kb″ is an integer of 0 to 7;
Lb″ is an integer of 0 to 7;
p″ is an integer of 0 to 4; and
q″ is an integer of 0 to 4.

7. A compound represented by formula (I): 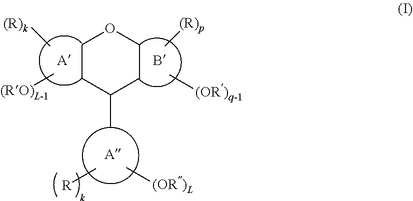
wherein
A′ and A″ represent the same aromatic ring;
B′ represents an aromatic ring;
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
L is an integer of 1 or more;
p is an integer of 0 or more;
q is an integer of 1 or more;
k is an integer of 0 or more; and
each —OR′ group is a hydroxy group, a crosslinkable group, or a dissociation group.

8. The compound according to claim 7, represented by formula (I′): 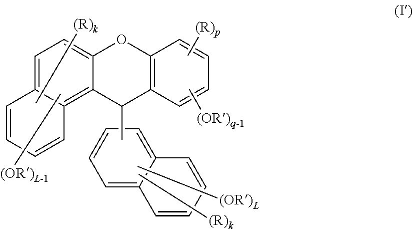
wherein
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
L is an integer of 1 or more;
p is an integer of 0 or more;
q is an integer of 1 or more;
k is an integer of 0 or more; and
each —OR′ group is a hydroxy group, a crosslinkable group, or a dissociation group.

9. A method for producing the compound according to claim 1, the method comprising a step of subjecting a phenol represented by the formula (1-1) and a aromatic aldehyde represented by the formula (2-1) to a condensation reaction, thereby obtaining a skeleton represented by the formula (3-1).

10. A resin having a constituent unit derived from the compound according to claim 1.

11. The resin according to claim 10, wherein the resin has a structure represented by the following formula (4): [symbol]
L₂-M [symbol] (4)
wherein L2 is a divalent group having 1 to 60 carbon atoms and M is a unit structure derived from a compound comprising a condensed skeleton of an aromatic compound represented by formula (1-1) and an aromatic aldehyde represented by formula (2-1): 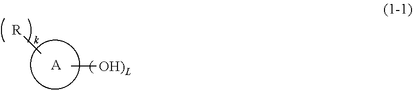
wherein
A represents an aromatic ring;
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
k is an integer of 0 or more; and
L is an integer of 1 or more, and 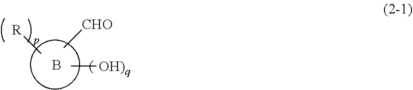
wherein
B represents an aromatic ring;
R is each independently an alkyl group having 1 to 30 carbon atoms and optionally having a substituent, an aryl group having 6 to 30 carbon atoms and optionally having a substituent, an alkenyl group having 2 to 30 carbon atoms and optionally having a substituent, an alkynyl group having 2 to 30 carbon atoms and optionally having a substituent, an alkoxy group having 1 to 30 carbon atoms and optionally having a substituent, a halogen atom, a nitro group, an amino group, a carboxylic acid group, a crosslinkable group, a dissociation group, or a thiol group;
the alkyl group, the aryl group, the alkenyl group, the alkynyl group, and the alkoxy group each optionally contain an ether bond, a ketone bond, or an ester bond;
p is an integer of 0 or more; and
q is an integer of 1 or more,
provided that at least one hydroxy group is bonded to a carbon atom adjacent to a carbon atom to which a formyl group is bonded.

12. A composition comprising the compound according to claim 1.

13. (canceled)

14. (canceled)

15. (canceled)

16. The composition according to claim 12, wherein the composition is used in film formation for lithography.

17. The composition according to claim 12, wherein the composition is used in film formation for resist.

18. The composition according to claim 12, wherein the composition is used in resist underlayer film formation.

19. (canceled)

20. A method for forming a resist pattern, comprising:
a photoresist layer formation step of forming a photoresist layer on a substrate using the composition according to claim 16; and
a development step of irradiating a predetermined region of the photoresist layer formed through the photoresist layer formation step with radiation for development.

21. (canceled)

22. A method for forming a resist pattern, comprising:
an underlayer film formation step of forming an underlayer film on a substrate using the composition according to claim 16;
a photoresist layer formation step of forming at least one photoresist layer on the underlayer film formed through the underlayer film formation step; and
a step of irradiating a predetermined region of the photoresist layer formed through the photoresist layer formation step with radiation for development.

23. A method for forming a circuit pattern, comprising:
an underlayer film formation step of forming an underlayer film on a substrate using the composition according to claim 16;
an intermediate layer film formation step of forming an intermediate layer film on the underlayer film formed through the underlayer film formation step;
a photoresist layer formation step of forming at least one photoresist layer on the intermediate layer film formed through the intermediate layer film formation step;
a resist pattern formation step of irradiating a predetermined region of the photoresist layer formed through the photoresist layer formation step with radiation for development, thereby forming a resist pattern;
an intermediate layer film pattern formation step of etching the intermediate layer film with the resist pattern formed through the resist pattern formation step as a mask, thereby forming an intermediate layer film pattern;
an underlayer film pattern formation step of etching the underlayer film with the intermediate layer film pattern formed through the intermediate layer film pattern formation step as a mask, thereby forming an underlayer film pattern; and
a substrate pattern formation step of etching the substrate with the underlayer film pattern formed through the underlayer film pattern formation step as a mask, thereby forming a pattern on the substrate.

24. A method for purifying the compound according to claim 1, comprising:
an extraction step in which extraction is carried out by bringing a solution containing the compound or resin, and an organic solvent that does not inadvertently mix with water into contact with an acidic aqueous solution.
